(19) United States
(12) Patent Application Publication
(10) Pub. No.: US 2015/0017156 A1
(43) Pub. Date: Jan. 15, 2015

(54) ESX-MEDIATED TRANSCRIPTION MODULATORS AND RELATED METHODS

(71) Applicants: Anna Mapp, Ann Arbor, MI (US); Quintin Pan, Columbus, OH (US)

(72) Inventors: Anna Mapp, Ann Arbor, MI (US); Quintin Pan, Columbus, OH (US)

(73) Assignees: THE OHIO STATE UNIVERSITY, Columbus, OH (US); THE REGENTS OF THE UNIVERSITY OF MICHIGAN, Ann Arbor, MI (US)

(21) Appl. No.: 14/344,069

(22) PCT Filed: Sep. 14, 2012
(86) PCT No.: PCT/US2012/055543

§ 371 Date: Sep. 26, 2014

Related U.S. Application Data
(60) Provisional application No. 61/535,843, filed on Sep. 16, 2011.

(51) Int. Cl. C07D 261/02; A61K 31/517; A61K 39/395; A61K 31/5377; A61K 31/42; C07K 16/28

(52) U.S. Cl. CPC C07D 261/02; A61K 31/42; C07K 16/2863; A61K 39/3955; A61K 31/5377; A61K 31/517; A61K 2039/505
USPC 424/133.1; 514/378; 424/142.1; 514/234.5; 514/266.24; 435/375; 435/29

(57) ABSTRACT

The present invention relates to gene regulation. In particular, the present invention provides small compounds capable of modulating ESX-mediated transcription and related methods of therapeutic and research use. In addition, the present invention provides methods for treating conditions associated with aberrant EGFR expression with ESX-mediated transcription modulators (e.g., ESX-mediated transcription inhibitors).

### CROSS-REFERENCE TO RELATED APPLICATIONS

This application claims priority to pending U.S. Provisional Patent Application No. _{61/535,843}, filed Sep. 16, 2011, the contents of which are incorporated by reference in its entirety.

### STATEMENT REGARDING FEDERALLY SPONSORED RESEARCH OR DEVELOPMENT

This invention was made with support under Grant No. CA140667 awarded by the National Institutes of Health. The government has certain rights in the invention.

### FIELD OF THE INVENTION

The present invention relates to gene regulation. In particular, the present invention provides small compounds capable of modulating ESX-mediated transcription and related methods of therapeutic and research use. In addition, the present invention provides methods for treating conditions associated with aberrant EGFR expression with ESX-mediated transcription modulators (e.g., ESX-mediated transcription inhibitors).

### BACKGROUND OF THE INVENTION

Head and neck squamous cell carcinomas (HNSCCs) represent the sixth most common cancers in the world, with about 500,000 new cases reported annually. This disease results in nearly 11,000 deaths each year in the United States alone. The most prevalent risk factors involved in the development of these highly aggressive malignancies, such as alcohol and tobacco use, betel nut chewing, and infection with the human papillomavirus have been long well recognized. However, the five-year survival rate after diagnosis for HNSCC remains low, approximately 50%, which is considerably lower than that for other cancers, such as those of colorectal, cervix and breast origin.

The poor prognosis of HNSCC patients is likely due to the fact that most patients are diagnosed at advanced disease stages, and often fail to respond to available treatment options. Moreover, the effects of the disease are highly disfiguring to the individual.

Accordingly, there is a desire to find a method of preventing the development of the disease.

### SUMMARY OF THE INVENTION

Epidermal growth factor receptor (EGFR) is elevated in over 90% of head and neck squamous cell carcinoma (HNSCC). However, results from clinical trials showed that a majority of HNSCC patients do not respond to anti-EGFR therapy. Resistance to EGFR inhibitors may be due to kinase-independent actions of EGFR and/or activation of another EGFR family member, Her2. Strategies to reduce EGFR and Her2 protein levels in concert may be an attractive approach to enhance the efficacy of current EGFR inhibitors.

In experiments conducted during the course of developing embodiments for the present invention, epithelial-restricted with serine box (ESX), a member of the ETS transcription factor family, was demonstrated to be elevated in primary HNSCC tumors and associated with increased EGFR and Her2 expression (p<0.05, n=16). In addition, shRNA-mediated knockdown of ESX in CAL27 cells resulted in decreased EGFR and Her2 protein levels and inhibited cell proliferation, invasion, and migration. Additionally, it was shown that ESX knockdown dramatically dampened EGFR promoter activity demonstrating that ESX directly regulates EGFR expression. Biphenyl isoxazolidine, a small molecule ESX mimic designed to inhibit ESX-mediated transcriptional activity, was shown to reduce EGFR and Her2 levels in CAL27 and SCC25 HNSCC cells. Biphenyl isoxazolidine inhibited cell proliferation with an IC50 of 46.8 μmol/L for CAL27 and 50.3 μmol/L for SCC25. Cell migration and invasion was dramatically decreased in HNSCC cells using sub-IC50 doses of biphenyl isoxazolidine. Moreover, biphenyl isoxazolidine enhanced the anti-proliferative effects of afatinib, gefitinib and lapatinib, three EGFR tyrosine kinase inhibitors. As such, the experiments conducted during the course of developing embodiments for the present invention indicated that ESX enhances EGFR expression through EGFR promoter activation revealing, for example, a novel mechanism of elevated EGFR levels in HNSCC. Moreover, the experiments indicate that inhibition of ESX to reduce EGFR and Her2 levels as an approach to enhance the response rate of HNSCC patients to current anti-EGFR therapeutics.

Accordingly, the present invention provides small compounds capable of modulating ESX-mediated transcription and related methods of therapeutic and research use. In addition, the present invention provides methods for treating conditions associated with aberrant EGFR expression with ESX-mediated transcription inhibitors.

In certain embodiments, the present invention provides methods for regulating ESX-mediated transcription of a gene of interest. In particular, the methods involve, for example, providing host cells expressing: ESX, an ESX transcription coactivator protein required for the ESX-mediated transcription of a gene of interest, and a gene of interest, and small molecules capable of binding with at least a portion of amino acid residues 137-146 (SWIIELLE) (SEQ ID NO:1) within ESX (e.g., thereby facilitating binding of the isoxazolidine compound within the respective region); delivering to the host cells an effective amount of the small molecules such that expression of the gene of interest is modified. In some embodiments, the host cells are ex vivo cells, in vivo cells, or in vitro cells. In some embodiments, the cells are cancer cells (e.g., cancer cells overexpressing ESX). In some embodiments, the cancer cells are HNSCC cells (e.g., HNSCC cells overexpressing ESX).

The methods are not limited to particular ESX transcription coactivator proteins. In some embodiments, the ESX transcription coactivator proteins include, but are not limited to, Med23 and Med15.

The methods are not limited to a particular gene of interest. In some embodiments, the gene of interest is ErbB2(Her2). In some embodiments, the gene of interest is EGFR.

The methods are not limited to particular small molecules capable of binding with at least a portion of amino acid residues 137-146 (SWIIELLE) (SEQ ID NO:1) within ESX (e.g., thereby facilitating binding of the isoxazolidine compound within the respective region). In some embodiments, the small molecules include isoxazolidine compounds. The small molecules are not limited to particular isoxazolidine compounds. In some embodiments, the isoxazolidine compounds are represented by the following formula:

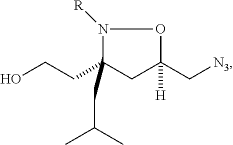

including salts, esters and prodrugs thereof, wherein R is a functional group that mimics at least a portion of the eight amino acid (137-SWIIELLE-146) (SEQ ID NO:1) α-helical region in ESX reported to mediate the interaction between ESX and Med23. In some embodiments, R is a functional group that mimics the effect of Tryptophan 138 of ESX. In some embodiments, R is a functional group that mimics the formation of a hydrophobic surface along an amphipathic helix within amino acids 137-146 of ESX. In some embodiments, R is selected from the group consisting of:

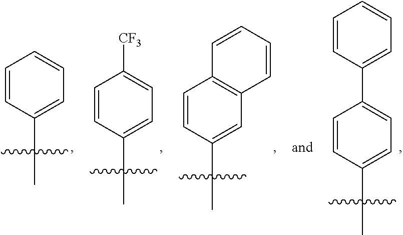

In some embodiments, the small molecules are selected from the group consisting of

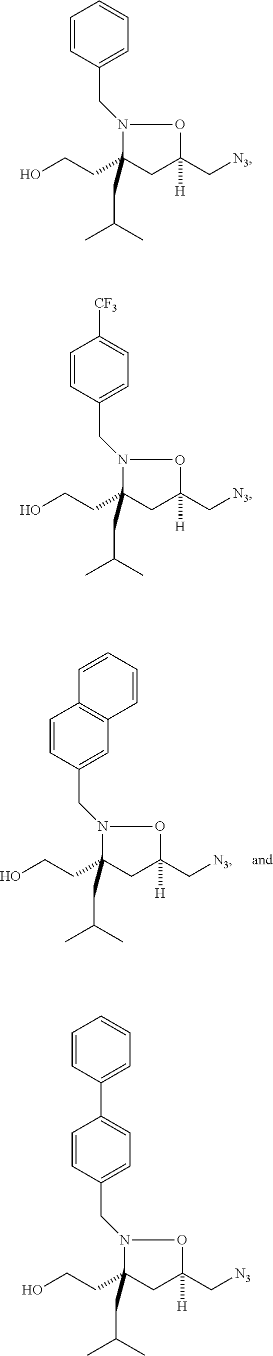 [SMILES: CC(C)C[C@]1(CCO)C[C@H](CN=[N+]=[N-])ON1Cc1ccccc1 ; CC(C)C[C@]1(CCO)C[C@H](CN=[N+]=[N-])ON1Cc1ccc(C(F)(F)F)cc1 ; CC(C)C[C@]1(CCO)C[C@H](CN=[N+]=[N-])ON1Cc1ccc2ccccc2c1 ; "and" ; CC(C)C[C@]1(CCO)C[C@H](CN=[N+]=[N-])ON1Cc1ccc(-c2ccccc2)cc1]

(biphenyl isoxazolidine).

In certain embodiments, the present invention provides methods for treating a subject having a disorder having elevated EGFR expression. The present invention is not limited to particular methods for treating disorders having elevated EGFR expression. In some embodiments the methods involve, for example, administering to the subject a pharmaceutical composition comprising an ESX-mediated transcription inhibitor. Any type of subject is contemplated for such methods (e.g., human, dog, cat, cow, ape, etc.).

The methods are not limited to a particular disorder having elevated EGFR expression. In some embodiments, the disorder is a cancer or cancer related disorder. In some embodiments, the cancer is HNSCC.

The methods are not limited to a particular type of ESX-mediated transcription inhibitor. In some embodiments, the ESX-mediated transcription inhibitor is an isoxazolidine compound. The methods are not limited to a particular isoxazolidine compound. In some embodiments, the isoxazolidine compound is represented by the following formula:

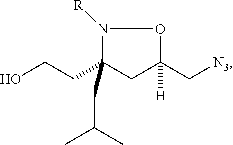

including salts, esters and prodrugs thereof, wherein R is a functional group that mimics at least a portion of the eight amino acid (137-SWIIELLE-146) (SEQ ID NO:1) α-helical region in ESX. In some embodiments, R is a functional group that mimics the effect of Tryptophan 138 of ESX. In some embodiments, R is a functional group that mimics the formation of a hydrophobic surface along an amphipathic helix within amino acids 137-146 of ESX. In some embodiments, R is selected from the group consisting of:

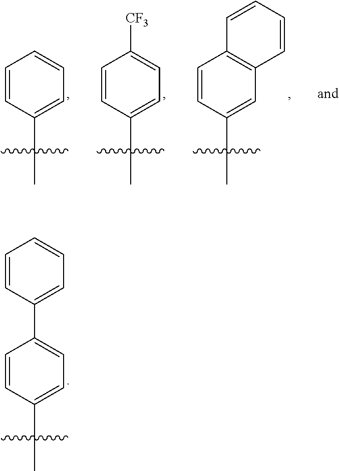

In some embodiments, the ESX-mediated transcription inhibitor is selected from the group consisting of

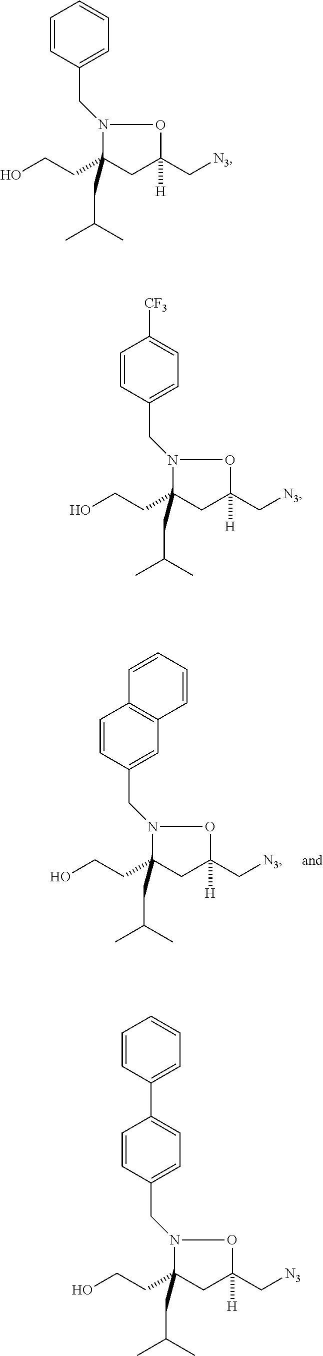 [SMILES: CC(C)C[C@]1(CCO)C[C@H](CN=[N+]=[N-])ON1Cc1ccccc1 ; CC(C)C[C@]1(CCO)C[C@H](CN=[N+]=[N-])ON1Cc1ccc(C(F)(F)F)cc1 ; CC(C)C[C@]1(CCO)C[C@H](CN=[N+]=[N-])ON1Cc1ccc2ccccc2c1 ; "and" ; CC(C)C[C@]1(CCO)C[C@H](CN=[N+]=[N-])ON1Cc1ccc(-c2ccccc2)cc1]

(biphenyl isoxazolidine).

Experiments conducted during the course of developing embodiments for the present invention demonstrated that biphenyl isoxazolidine enhanced the anti-proliferative effects of afatinib, gefitinib and lapatinib, three EGFR tyrosine kinase inhibitors. Indeed, the experiments indicate inhibition of ESX to reduce EGFR and Her2 levels as an approach to enhance the response rate of HNSCC patients to current anti-EGFR therapeutics. Accordingly, in some embodiments, the ESX-mediated transcription inhibitor is co-administered with a therapeutic agent known for treating a disorder having elevated EGFR expression. For example, in some embodiments, the methods further comprise co-administering to the subject a therapeutic agent selected from the group consisting of an EGFR monoclonal antibody inhibitor (e.g., cetuximab, panitumumab, zalutumubab, nimotuzumab, matuzumab), a tyrosine kinase inhibitor (e.g., afatinib, gefitinib, erlotinib, lapatinib), and/or any therapeutic agents known/used for treating EGFR related disorders (e.g., cancer (e.g., HNSCC, lung cancer, colorectal cancer) (e.g., AP26113 (e.g., ARIAD Pharmaceuticals), potato carboxypeptidase inhibitors (see, e.g., Blanco-Aparicio, et al 1998).

Experiments conducted during the course of developing embodiments for the present invention demonstrated that combinations of transcriptional inhibitors of erbB2 and existing therapeutic agents that target erbB2 activity and lifetime lead to a synergistic increase in activity, with dose reductions as high as 30 fold compared to individual agents (see Example 5). Accordingly, in certain embodiments, the present invention provides methods for treating a subject having a disorder having elevated erbB2 expression. The present invention is not limited to particular methods for treating disorders having elevated erbB2 expression. In some embodiments the methods involve, for example, co-administering to the subject an effective amount of an ESX-mediated transcription inhibitor, and one or more agents known to target the activity and lifetime of the erbB2 oncoprotein. Any type of subject is contemplated for such methods (e.g., human, dog, cat, cow, ape, etc.).

The methods are not limited to a particular disorder having elevated erbB2 expression. In some embodiments, the disorder is a cancer or cancer related disorder (e.g., breast cancer, stomach cancer, ovarian cancer, endometrial carcinoma (see, e.g, Santin, 2008). In some embodiments, the cancer is breast cancer.

The methods are not limited to a particular type of ESX-mediated transcription inhibitor. In some embodiments, the ESX-mediated transcription inhibitor is an isoxazolidine compound. The methods are not limited to a particular isoxazolidine compound. In some embodiments, the isoxazolidine compound is represented by the following formula:

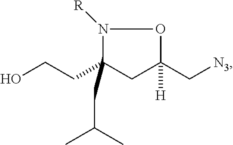

including salts, esters and prodrugs thereof, wherein R is a functional group that mimics at least a portion of the eight amino acid (137-SWIIELLE-146) (SEQ ID NO:1) α-helical region in ESX. In some embodiments, R is a functional group that mimics the effect of Tryptophan 138 of ESX. In some embodiments, R is a functional group that mimics the formation of a hydrophobic surface along an amphipathic helix within amino acids 137-146 of ESX. In some embodiments, R is selected from the group consisting of:

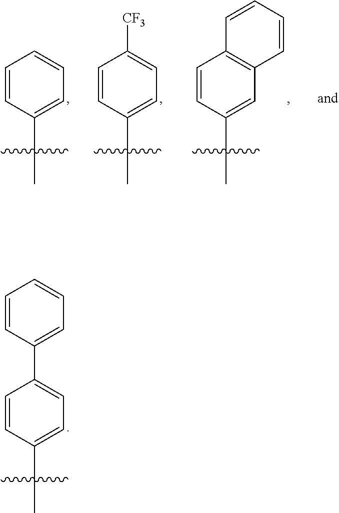

In some embodiments, the ESX-mediated transcription inhibitor is selected from the group consisting of

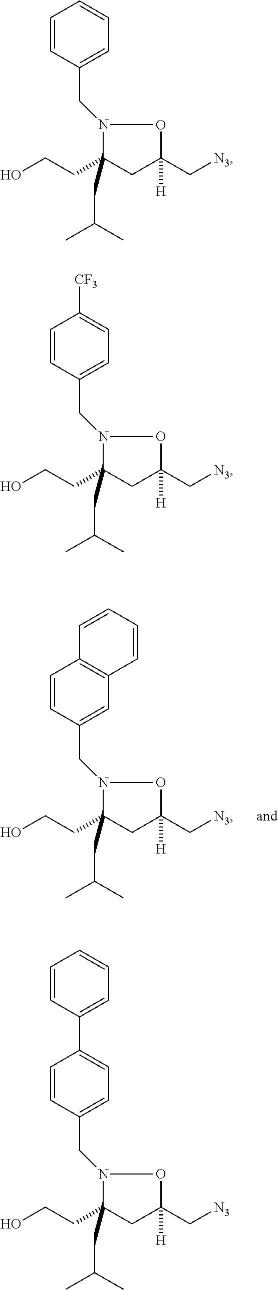

(biphenyl isoxazolidine).

The methods are not limited to particular agents known to target the activity and lifetime of the erbB2 oncoprotein. In some embodiments, the agent is a tyrosine kinase inhibitor (e.g., afatinib, gefitinib, erlotinib, lapatinib (see Example 5). In some embodiments, the agent is an anti-tumor antibiotic (e.g., benzoquinone ansamycin antibiotics (e.g., geldanamycin (see, e.g., Bedin, 2004) (see Example 5), 17-N-Allylamino-17-demethoxygeldanamycin (17-AAG), 17-Dimethylaminoethylamino-17-demethoxygeldanamycin (17-DMAG))).

In certain embodiments, the present invention provides methods for identifying ESX-mediated transcription modulators, comprising, for example, providing i) host cells expressing ESX, a gene whose transcription is regulated by ESX, and ESX-mediated transcription coactivating compounds required for the ESX-mediated transcription of the gene of interest, and ii) a potential ESX-mediated transcription modulator, delivering to the host cells an effective amount of the potential ESX-mediated transcription modulator, and detecting changes in ESX-mediated transcription, wherein inhibition in ESX-mediated transcription indicates the potential ESX-mediated transcription modulator is an ESX-mediated transcription inhibitor, wherein enhancement in ESX-mediated transcription indicates the potential ESX-mediated transcription modulator is an ESX-mediated transcription enhancer.

### BRIEF DESCRIPTION OF THE DRAWINGS

FIG. 1 shows that ESX is elevated and associated with EGFR and Her2 in HNSCC. Sixteen primary tumors were collected from HNSCC patients at the time of surgical resection between 1997 and 2000. All tissues were diagnosed histologically as HNSCC by a board certified pathologist. Written informed consent, as required by the institutional review board, was obtained from all patients. Collected samples were stored immediately in liquid nitrogen at −80° C. until analysis. Total RNA was isolated from the frozen tumors with TRIzol (Invitrogen, Carlsbad, Calif.). Expression of ESX, EGFR, and Her2 were determined using the Applied Biosystems 7900HT Fast Real-Time PCR System with validated TaqMan gene expression assays (Applied Biosystems, Foster City, Calif.). Gene expression was normalized to GAPDH using the ΔΔCt method. FIG. 1A. ESX expression in primary HNSCC tumors. Patients with the highest 8 ESX expression were binned into the high ESX group and patients with the lowest 8 ESX expression were binned into the low ESX group. p=0.0012, n=16. FIG. 1B. ESX is associated with EGFR. p<0.05, n=16. FIG. 1C. ESX is associated with Her2. p<0.05, n=16. FIG. 1D. ESX is elevated in HNSCC cell lines Immunoblot analyses using an ESX-specific antibody (GenWay Biotech, San Diego, Calif.), EGFR-specific antibody (Cell Signaling Technology, Danvers, Mass.), a Her2-specific antibody (Santa Cruz Biotechnology, Santa Cruz, Calif.), and a GAPDH-specific antibody (Sigma, St. Louis, Mo.) were performed.

FIG. 2 shows that Genetic knockdown of ESX inhibits EGFR levels and promoter activity and reduces in vitro tumorigenicity in HNSCC. A. ESX knockdown reduces EGFR levels in CAL27 cells. CAL27 cells were transduced with shRNA-control or shRNA-ESX (pGIPZ Lentiviral shRNAmir; Open Biosystems, Huntsville, Ala.) and selected in antibiotics to generate polyclonal CAL27/shRNA-control and CAL27/shRNA-ESX cells. Cell lysates were prepared and determined for ESX, EGFR, and Her2 protein levels. B. EGFR promoter activity. CAL27/shRNA-control and CAL27/shRNA-ESX cells were transiently transfected with an EGFR promoter-Firefly luciferase vector and a Renilla luciferase vector. After 24 hours, cell lysates were prepared and measured for Firefly and Renilla luciferase activity. EGFR promoter activity was normalized to Renilla luciferase activity to control for transfection efficiency. Data is presented as mean±SEM. * p<0.001, n=9. C. Cell proliferation. CAL27/shRNA-control and CAL27/shRNA-ESX cells were plated and allowed to proliferate for 48 hours. Cell proliferation was assessed using the CCK-8 reagent to detect metabolic active cells (Dojindo Inc., Gaithersburgh, Md.). The absorbance at 450 nm was quantitated using a microplate reader (Molecular Devices, Sunnyvale, Calif.). Data is presented as mean±SEM. * p<0.005, n=3. D. Cell invasion. CAL27/shRNA-control and CAL27/shRNA-ESX cells were harvested, and resuspended in serum-free medium. An aliquot (1×105 cells) of the prepared cell suspension was added to the top chamber and 10% FBS was added to the bottom chamber. After 24 hours, non-invading cells were gently removed from the interior of the inserts with a cottontipped swab. Invasive cells were visualized with fluorescence microscopy. A representative field for each experimental condition is presented. E. Cell migration. Cells were seeded and allowed to grow until confluence. Confluent monolayers were scratched using a sterile pipette tip, washed, and incubated in complete medium. A representative field for each experimental condition at 0 hour and 10 hours is presented.

FIG. 3 shows that biphenyl isoxazolidine reduces EGFR and inhibits cell proliferation, invasion, and migration in HNSCC. A. EGFR and Her2 levels. CAL27 and SCC25 cells were treated with biphenyl isoxazolidine for 24 hours. Cell lysates were prepared and determined for EGFR and Her2 protein levels by immunoblot analyses. B. Cell proliferation. Cells were untreated or treated with biphenyl isoxazolidine (ESXm) for 24 hours. Cell proliferation was assessed using the CCK-8 reagent to detect metabolic active cells. Dose-response curves and IC50 values were generated using GraphPad Prism 4.0 (GraphPad Software, La Jolla, Calif.). IC50 was 46.8 μmol/L for CAL27 and 50.3 μmol/L for SCC25. C. Cell invasion. Cell invasion was determined as described from the cell invasion assay kit (Chemicon International, Temecula, Calif.). Cells were treated with biphenyl isoxazolidine for 24 hours, harvested, and re-suspended in serumfree medium. An aliquot (1×105 cells) of the prepared cell suspension was added to the top chamber and 10% FBS was added to the bottom chamber. After 24 hours, non-invading cells were gently removed from the interior of the inserts with a cotton-tipped swab. Invasive cells were stained and visualized. A representative field for each experimental condition is presented. D. Cell migration. Cell migration was determined using the wound healing assay. Cells were seeded and allowed to grow until confluence. Confluent monolayers were treated with biphenyl isoxazolidine for 24 hours, scratched using a sterile pipette tip, washed, and incubated in complete medium. A representative field for each experimental condition at 0 hour and 24 hours is presented.

FIG. 4 shows that biphenyl isoxazolidine potentiates the anti-proliferative effects of gefitinib and lapatinib. CAL27 and SCC25 cells were treated with gefitinib, an EGFR TKI, or lapatinib, a dual EGFR/Her2 TKI, at various concentrations with and without an IC50 dose of biphenyl isoxazolidine (ESXm) (FIG. 4A CAL27/gefitinib; FIG. 4B SCC25/gefitinib; FIG. 4C CAL27/lapatinib; FIG. 4D SCC25/lapatinib). Cell proliferation was assessed using the CCK-8 reagent to detect metabolic active cells. Dose-response curves and IC50 values were generated using GraphPad Prism 4.0 (GraphPad Software, La Jolla, Calif.). IC50 was 26.3 μmol/L for gefitinib and 11.8 μmol/L for lapatinib in CAL27 cells. IC50 was 70.7 μmol/L for gefitinib and 11.9 μmol/L for lapatinib in SCC25 cells. The combination treatment with IC50 ESXm and IC50 gefitinib decreased cell proliferation by 90.1% and 97% in CAL27 and SCC25 cells, respectively. The combination treatment with IC50 ESXm and IC50 lapatinib decreased cell proliferation by 90.4% and 94.2% in CAL27 and SCC25 cells, respectively.

FIG. 5 is a schematic of erbB2 pathway and points of small molecule intervention (see, e.g., Roe, 1999; Isaacs, 2003; Petrov, 2006) Combinations that inhibit both the transcription of erbB2 and the lifetime or activity of the mature protein have a synergistic increase in activity against erbB2 driven cancer cells.

FIG. 6 shows a) The dose effect curves for biphenyl isoxazoline (i1) as a single agent and a 50:1 combination of biphenyl isoxazoline:geldanamcyin after 3 days of dosing. b) The IC50s of fixed dose ratios of biphenyl isoxazoline and geldanamycin were measured in SKBR3 cells after 3 days of dosing and plotted on an isobologram. c) The % effect (100-% growth) for the indicated doses for the 3 day dosing period of a growth timecourse (FIG. 7c-d). Predicted additivity was calculated as indicated in the SI. d) The IC50s from (b) were compared to IC50s for the same combinations in IMR90 cells (FIG. 11a-c) and the resulting ratios were plotted as shown, normalized to the effects of biphenyl isoxazoline and geldanamycin as single agents. Error bars indicate error compounded from one standard deviation of experiments performed in triplicate. For all other experiments, error bars indicate one standard deviation from experiments performed in triplicate unless noted otherwise.

FIG. 7 shows the effects of i1, geldanamycin, and combinations in SkBr3 cells. a) A combination of it (biphenyl isoxazolidine) and geldanamycin is more effective at reducing levels of erbB2 than either agent in isolation (p<0.01). Levels of erbB2 were normalized to the total protein concentration for each well. Error bars represent the standard deviation of this ratio. b) Comparison of the IC₅₀s (in SkBr3 cells) of i1, geldanamycin, and combinations using an isobologram indicate that combinations provide a synergistic reduction in the required dose of each compound. The combination index, with compounded error from the isobologram, is shown. Error bars on the isobologram represent compounded standard error of the IC₅₀s. c) The effects of i1 (40 nM), Geldanamycin (8 nM) and a combination of the two on ErbB2+ (SkBr3) cells was evaluated over 9 days of treatment. Error bars are 1 standard deviation d) Evaluation of individual timepoints indicates indicate a synergistic increase in effect for the combination. % effect is calculated by normalizing the % viability to that of the control group for each timepoint and subtracting from 100. Predicted additivity is calculated according to the multiplicative method of Bliss as described in the supporting information. e) Dose-effect curves used to generate isobolograms for i1:geldanamycin concentrations, displayed as a function of geldanamycin concentration. f) Dose-effect curves used to generate isobolograms for i1:geldanamycin concentrations, displayed as a function of i1 concentration.

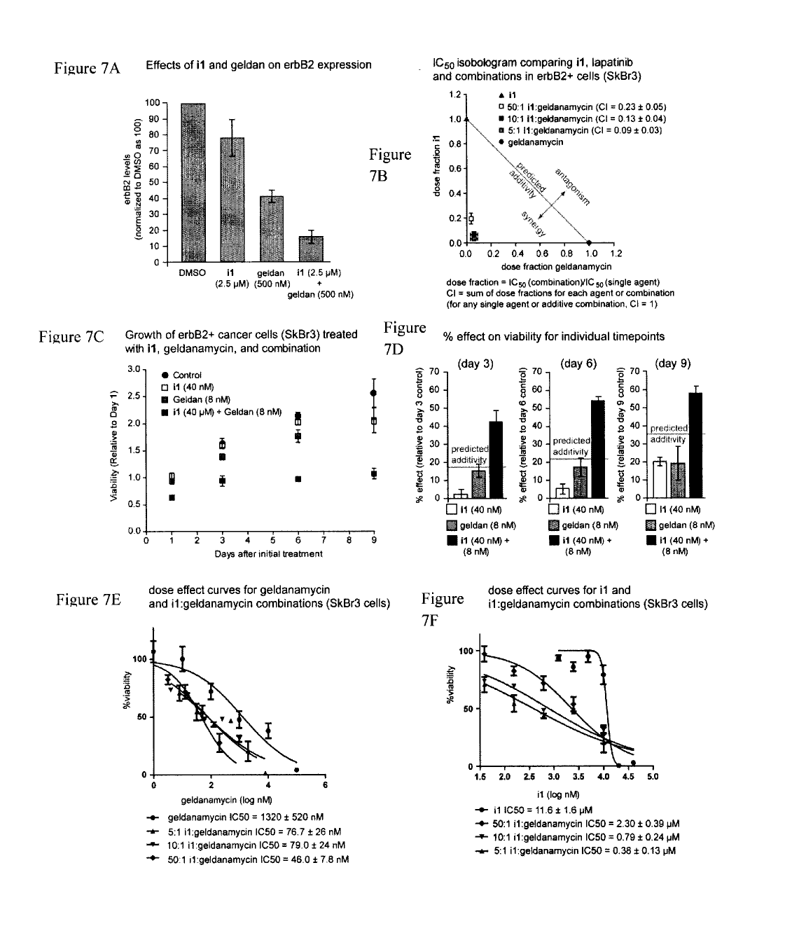

FIG. 8 shows a) The dose effect curves for biphenyl isoxazoline (i1) as a single agent, and a 500:1 combination of biphenyl isoxazoline:lapatinib after 2 days of dosing. b) The IC50's of fixed dose ratios of biphenyl isoxazoline and lapatinib were measured in SKBR3 cells after 2 days of dosing and plotted on an isobologram. c) The % effect (100-% growth) for the indicated doses for the 3 day dosing period of a growth timecourse. Predicted additivity was calculated as indicated in the SI. d) The IC50's from (b) were compared to IC50's for the same combinations in IMR90 cells (FIG. 9e) and the resulting ratios were plotted as shown, normalized to the effects of biphenyl isoxazoline and lapatinib as single agents. Error bars indicate error compounded from one standard deviation of experiments performed in triplicate. For all other experiments, error bars indicate one standard deviation from experiments performed in triplicate unless noted otherwise.

FIG. 9 shows the effects of i1, lapatinib, and combinations. a) After 1 day, a combination of i1 and lapatinib is more effective at reducing levels of active (phosphorylated) erbB2 than either agent in isolation (p<0.05). Levels of erbB2 and p-erbB2 were normalized to the total protein concentration for each well. Error bars represent the standard deviation of this ratio. b) Comparison of the IC₅₀s (in SkBr3 cells) of i1, lapatinib, and combinations using an isobologram indicate that combinations provide a synergistic reduction in the required dose of each compound. The combination index, with compounded error from the isobologram, is shown. Error bars on the isobologram represent compounded standard error of the IC₅₀s. c)Dose-effect curves used to generate isobolograms for i1:lapatinib concentrations, displayed as a function of lapatinib concentration. 500:1 i1:lapatinib is from a separate plate in which the lapatinib IC₅₀ was 48±17 nM. d) Dose-effect curves used to generate isobolograms for i1:lapatinib concentrations, displayed as a function of i1 concentration. 500:1 i1:lapatinib is from a seperate plate in which the i1 IC50 was 13.7±1.0 μM. e) Dose-effect curves used to generate isobolograms for i1:lapatinib concentrations in IMR90 cells, displayed as a function of i1 concentration. f) Dose-effect curves used to generate isobolograms for i1:lapatinib concentrations in IMR90 cells, displayed as a function of lapatinib concentration. All experiments were carried out in triplicate and error bars represent standard deviation unless otherwise indicated.

FIG. 10 shows the effects of i1, erlotinib, and combinations. a) Comparison of the IC50s after two days of treatment with i1, erlotinib, and combinations using an isobologram indicates that combinations cause an approximately additive reduction in the required dose of each compound, but this analysis is complicated by the tendency of erlotinib to precipitate out of cell culture media at higher concentrations, which are the concentrations at which synergy would be expected. Error bars represent compounded standard error. b) Dose-effect curves used to generate isobolograms for i1:erlotinib concentrations, displayed as a function of i1 concentration. c) Dose-effect curves used to generate isobolograms for i1:erlotinib concentrations, displayed as a function of erlotinib concentration. d) The effects of i1 (10 μM), erlotinib (2 μM) and a combination of the two on ErbB2+(SkBr3) cells was evaluated over 9 days of treatment, with no significant increase in effect. e) Evaluation of individual timepoints for the combination. % effect is calculated by normalizing the % viability to that of the control group for each timepoint and subtracting from 100. Predicted additivity is calculated according to the multiplicative method of Bliss as described in the supporting information. All experiments were carried out in triplicate and error bars represent standard deviation unless otherwise indicated.

FIG. 11 shows the effects of i1, geldanamycin, and combinations in IMR90 cells. Comparison of the IC₅₀s (in IMR90 cells) of i1, geldanamycin, and combinations using an isobologram indicate that combinations do not provide a synergistic reduction in the required dose of each compound. Error bars represent compounded standard error. b) Dose-effect curves used to generate isobolograms for i1:geldanamycin concentrations in IMR90 cells, displayed as a function of geldanamycin concentration. c) Dose-effect curves used to generate isobolograms for i1:geldanamycin concentrations, displayed as a function of i1 concentration.

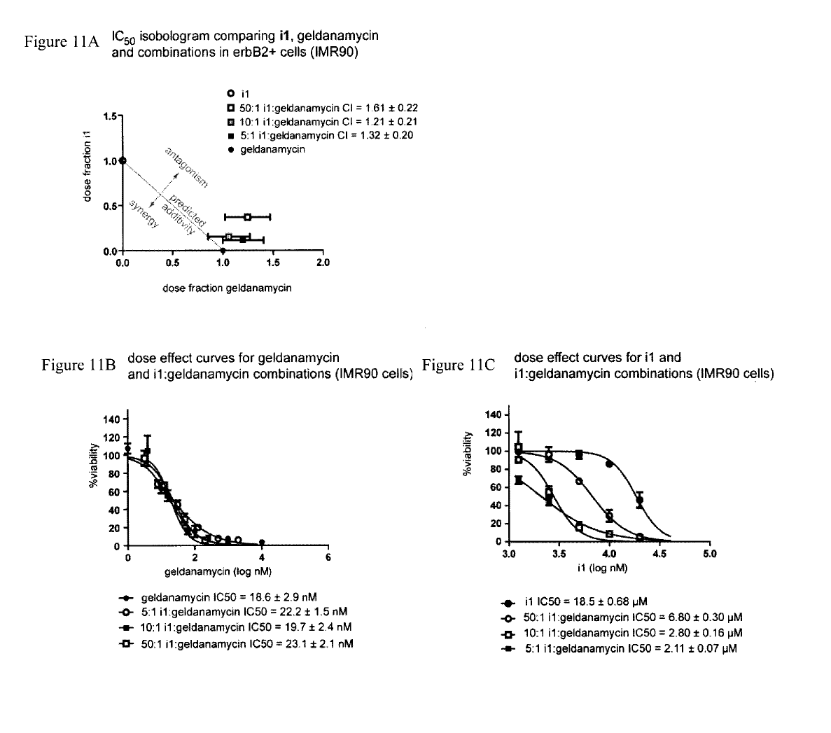

FIG. 12 shows anti-tumor efficacy of biphenyl isoxazolidine as single agent and in combination with afatinib, an irreversible EGFR/Her2 tyrosine kinase inhibitor, as assessed in a xenograft model of HNSCC. CAL27 HNSCC cells (1×10⁶) were implanted into the flank of 8-week-old athymic nude mice and tumors were allowed to develop without treatment. At 3 weeks post-tumor cell implantation, mice with established tumors were randomly assigned to four treatment arms; vehicle, biphenyl isoxazolidine (100 μg; 5× week; intratumoral injection), afatinib (20 mg/kg; 5× week; oral gavage), or biphenyl isoxazolidine and afatinib (see FIG. 12). As shown in FIG. 12, single-agent biphenyl isoxazolidine inhibited tumor growth by 51% (n=10, p<0.05) and single-agent afatinib suppressed tumor growth by 87% (n=10, p<0.01). The combination of biphenyl isoxazolidine and afatinib was the most active and blocked tumor growth by 94% (n=10. p<0.01). Mean tumor volume was 2.1-fold higher for the single-agent afatinib arm compared to the combination treatment arm (43 mm³ vs. 20 mm³) The anti-tumor efficacy of the combination treatment arm was statistically superior to either single-agent biphenyl isoxazolidine (p<0.01) or single-agent afatinib (p<0.01).

### DEFINITIONS

To facilitate an understanding of the invention, the following terms have the meanings defined below.

The term “host cell” or “cell” refers to any cell which is used in any of the methods of the present invention and may include prokaryotic cells, eukaryotic cells, yeast cells, bacterial cells, plant cells, animal cells, such as, reptilian cells, bird cells, fish cells, mammalian cells. Preferred cells include those derived from humans, dogs, cats, horses, cattle, sheep, pigs, llamas, gerbils, squirrels, goats, bears, chimpanzees, mice, rats, rabbits, etc. The term cells includes transgenic cells from cultures or from transgenic organisms. The cells may be from a specific tissue, body fluid, organ (e.g., brain tissue, nervous tissue, muscle tissue, retina tissue, kidney tissue, liver tissue, etc.), or any derivative fraction thereof. The term includes healthy cells, transgenic cells, cells affected by internal or exterior stimuli, cells suffering from a disease state or a disorder, cells undergoing transition (e.g., mitosis, meiosis, apoptosis, etc.), etc. The term also refers to cells in vivo or in vitro (e.g., the host cell may be located in a transgenic animal or in a human subject).

As used herein, the terms “host” and “subject” refer to any animal, including but not limited to, human and non-human animals (e.g. rodents, arthropods, insects (e.g., Diptera), fish (e.g., zebrafish), non-human primates, ovines, bovines, ruminants, lagomorphs, porcines, caprines, equines, canines, felines, ayes, etc.), that is studied, analyzed, tested, diagnosed or treated. As used herein, the terms “host” and “subject” are used interchangeably.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The term “promoter region” refers to the 5′ flanking region of a gene and may contain regulatory sequences such as promoters and enhancers that control or influence the transcription of the gene. The 3′ flanking region may contain sequences that direct the termination of transcription, post-transcriptional cleavage and polyadenylation.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, “expression” refers to the process by which nucleic acid is transcribed into mRNA and translated into peptides, polypeptides, or proteins. “Expression” may be characterized as follows: a cell is capable of synthesizing many proteins. At any given time, many proteins which the cell is capable of synthesizing are not being synthesized. When a particular polypeptide, coded for by a given gene, is being synthesized by the cell, that gene is said to be expressed. In order to be expressed, the DNA sequence coding for that particular polypeptide must be properly located with respect to the control region of the gene. The function of the control region is to permit the expression of the gene under its control. As used herein, the term “expression vector” includes vectors capable of expressing DNA or RNA fragments that are in operative linkage with regulatory sequences, such as promoter regions, that are capable of effecting expression of such DNA or RNA fragments. Thus, an expression vector refers to a recombinant DNA or RNA construct, such as a plasmid, a phage, recombinant virus or other vector that, upon introduction into an appropriate host cell, results in expression of the cloned DNA or RNA. Appropriate expression vectors are well known to those of skill in the art and include those that are replicable in eukaryotic cells and/or prokaryotic cells and those that remain episomal or may integrate into the host cell genome.

The term “gene transcription” as it is used herein means a process whereby one strand of a DNA molecule is used as a template for synthesis of a complementary RNA by RNA polymerase.

The term “pharmaceutical composition” refers to the combination of an active agent with a carrier, inert or active, making the composition especially suitable for diagnostic or therapeutic use in vivo or ex vivo.

The term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

The term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while sometime not in themselves pharmaceutically acceptable, may be employed in the preparation of salts useful as intermediates in obtaining the compounds of the invention and their pharmaceutically acceptable acid addition salts. Examples of bases include alkali metals (e.g., sodium) hydroxides, alkaline earth metals (e.g., magnesium), hydroxides, ammonia, and compounds of formula NW₄⁺, wherein W is C₁₋₄ alkyl, and the like. Examples of salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “effective amount” refers to the amount of a compound sufficient to effect beneficial or desired results. An effective amount can be administered in one or more administrations, applications or dosages and is not limited or intended to be limited to a particular formulation or administration route.

The term “second agent” refers to a therapeutic agent other than the isoxazolidine compounds in accordance with the present invention. In certain instances, the second agent is an anti-proliferative agent.

The term “co-administration” refers to the administration of at least two agent(s) (e.g., a compound of the present invention) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “combination therapy” includes the administration of an isoxazolidine compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### DETAILED DESCRIPTION

The ETS transcription factor family is intimately involved in tumorigenesis through direct regulation of genes critical for angiogenesis, apoptosis, invasion, and proliferation (see, e.g., Oikawa and Yamada, 2003). Epithelial-restricted with serine box (ESX), a member of the ETS transcription factor family, is exclusively expressed in terminally differentiated epithelial cells suggesting that ESX may play a role in controlling cell differentiation (see, e.g., Cabral et al.; Oettgen et al.). ESX was reported to be over-expressed in breast cancer, in part, through gene amplification (see, e.g., Liu et al., 1992). In addition, ESX binds to an ESX response element to transactivate the Her2 promoter (see, e.g., Chang et al., 1997). Ectopic expression of ESX is sufficient to transform MCF12A mammary epithelial cells resulting in epidermal growth factor-independent proliferation, increase cell invasion and motility, and anchorage-independent growth (Schedin et al., 2004).

ESX interacts with multiple coactivator proteins to regulate gene transcription. Med23, the most well characterized ESX coactivator, interacts with ESX to regulate Her2 transcription. An eight amino acid (137-SWIIELLE-146) (SEQ ID NO:1) α-helical region in ESX was reported to mediate the interaction between ESX and Med23 (see, e.g., Asada et al., 2002). Tryptophan 138 was shown to be essential for the specificity of the ESX-Med23 interaction (see, e.g., Asada et al., 2002). In addition, NMR spectroscopy suggests that W138 along with 1139, 1140, L142, and L143 form a hydrophobic surface along an amphipathic helix that interacts with Med23 (see, e.g., Asada et al., 2002). The binding interaction between ESX and Med23 was shown to be disrupted with a small molecule α-helix mimic of ESX, wrenchnolol (Shimogawa et al., 2004). Wrenchnolol decreased Her2 expression and inhibited cell proliferation of SKBR3 Her2-positive breast carcinoma cells (see, e.g., Shimogawa et al., 2004). Recently, isoxazoline novel α-helix ESX mimics (e.g., biphenyl isoxazolidine) were designed and synthesized to block the interaction between ESX-Med23 (see, e.g., Lee et al., 2009). Similar to wrenchnolol, isoxazoline novel α-helix ESX mimics (e.g., biphenyl isoxazolidine) decreased Her2 expression and inhibited cell proliferation of SKBR3 cells (see, e.g., Lee et al., 2009). These two studies showed that targeting the ESX-Med23 interaction is feasible and moreover, demonstrated that inhibition of transcription factor activation with small molecules is a novel and promising avenue for anti-cancer drug development.

Head and neck squamous cell carcinoma (HNSCC) is the sixth most common cancer with an annual incidence of approximately 600,000 cases worldwide (see, e.g., Kamangar et al., 2006). A well recognized genetic alteration in HNSCC is the dysregulation of epidermal growth factor receptor (EGFR). EGFR is almost universally over-expressed and elevated EGFR expression is associated with inferior clinical outcome in HNSCC patients (see, e.g., Nicholson et al., 2001; Rubin Grandis et al., 1998). Although elevated EGFR is a frequent event, only a small proportion, around 5-15%, of HNSCC patients responds to single-agent anti-EGFR therapy suggesting that blocking EGFR tyrosine kinase-dependent activity and/or downstream signaling is insufficient for optimal clinical response (see, e.g., Choong and Cohen, 2006).

A potential explanation for the low response rate to EGFR inhibitors may be due to the kinase-independent actions of EGFR A recent study showed that EGFR mediates cell survival by controlling autophagy independent of EGFR kinase activity (see, e.g., Weihua et al., 2008). EGFR can translocate from the cell membrane to the nucleus to regulate the transcription of genes involved in cell proliferation and survival (see, e.g., Lo and Hung, 2006; Wang and Hung, 2009). Alternatively, resistance to EGFR inhibitors may be due to a compensatory mechanism resulting in activation of other EGFR family members, in particular Her2 and Her3 (see, e.g., Erjala et al., 2006).

Experiments conducted during the course of developing embodiments for the present invention showed that EGFR expression is mediated by ESX, and that ESX is over-expressed and regulates EGFR expression in HNSCC. In addition, it was shown that isoxazoline novel α-helix mimics of ESX (e.g., biphenyl isoxazolidine) are active anti-cancer therapeutics and potentiated the anti-proliferative effects of EGFR TKIs. These results reveal that targeting ESX is a novel approach to enhance the efficacy of anti-EGFR therapeutics in HNSCC.

Accordingly, the present invention provides small molecules (e.g., compounds) capable of inhibiting ESX-mediated transcription and their therapeutic and/or research uses. Exemplary compositions and methods of the present invention are described in more detail in the following sections: I. ESX-Mediated Transcription Modulators; II. Methods for Identifying ESX-Mediated Transcription Modulators; III. Methods for Regulating ESX-Mediated Gene Transcription; IV. ESX-Mediated Transcription Based Therapeutics and Research Applications; V. Pharmaceutical Compositions; and VI. Other Embodiments.

### I. ESX-Mediated Transcription Modulators

ESX interacts with multiple coactivator proteins to regulate gene transcription. For example, Med23, the most well characterized ESX coactivator, interacts with ESX to regulate Her2 transcription. Experiments conducted during the course of developing embodiments for the present invention showed that ESX interacts (e.g., binds) with Med23 to regulate EGFR gene transcription. In addition, the interaction between ESX and Med23 is known to occur at an eight amino acid (137-SWIIELLE-146) (SEQ ID NO:1) α-helical region within ESX (see, e.g., Asada et al., 2002). Experiments conducted during the course of developing embodiments for the present invention further demonstrated that such ESX-mediated EGFR transcription can be inhibited through attenuating the binding between ESX and Med23 with small molecules (e.g., small molecules configured to bind the eight amino acid (137-SWIIELLE-146) (SEQ ID NO:1) α-helical region within ESX (e.g., the Med23/ESX interaction site within ESX)). Accordingly, the present invention provides ESX-mediated transcription modulators. The present invention is not limited to particular types or kinds of ESX-mediated transcription modulators. In some embodiments, the ESX-mediated transcription modulators are ESX-mediated transcription inhibitors. In some embodiments, the ESX-mediated transcription modulators are ESX-mediated transcription enhancers.

The present invention is not limited to particular types of ESX-mediated transcription inhibitors or ESX-mediated transcription enhancers. In some embodiments, the present invention provides small molecules capable of inhibiting or enhancing ESX-mediated transcription. The present invention is not limited to particular types of small molecules capable of inhibiting or enhancing ESX-mediated transcription. In some embodiments, such small molecules are capable of binding the locations within ESX where such coactivator proteins are known to interact (e.g., bind) with ESX in regulating gene transcription. Examples of such coactivators include, but are not limited to, Med23 (see, e.g., Asada et al., 2002; Lee et al., 2009), Tra1, and Med15.

In some embodiments, small molecules are provided that inhibit ESX-mediated transcription through binding the eight amino acid (137-SWIIELLE-146) (SEQ ID NO:1) α-helical region within ESX (e.g., the Med23/ESX interaction site within ESX) (e.g., thereby attenuating ESX/Med23 binding). The small molecules are not limited to a particular manner or structure that facilitate such interacting (e.g., binding) with the locations within ESX where Med23 is known to interact with ESX in regulating gene transcription. In some embodiments, wrenchnolol, and variants thereof, is provided (see, e.g., Shimogawa et al., 2004) as a small molecule capable of interacting (e.g., binding) within the eight amino acid (137-SWIIELLE-146) (SEQ ID NO:1) α-helical region within ESX (e.g., the Med23/ESX interaction site within ESX) (e.g., thereby attenuating ESX/Med23 binding). In some embodiments, the structure of the small molecule is such that it is able to mimic at least a portion of the eight amino acid (137-SWIIELLE-146) (SEQ ID NO:1) α-helical region in ESX reported to mediate the interaction between ESX and Med23 (see, e.g., Asada et al., 2002; Lee et al., 2009). In some embodiments, the structure of the small molecule is such that it is able to mimic the effect of Tryptophan 138 within ESX known to be essential for the specificity of the ESX-Med23 interaction (see, e.g., Asada et al., 2002). In some embodiments, the structure of the small molecule is such that it is able to mimic the formation of a hydrophobic surface along an amphipathic helix within amino acids 137-146 of ESX that interacts with Med23.

In some embodiments, small molecules that inhibit ESX-mediated transcription through interacting (e.g., binding) with at least a portion of the eight amino acid (137-SWIIELLE-146) (SEQ ID NO:1) α-helical region within ESX (e.g., the Med23/ESX interaction site within ESX) (e.g., thereby attenuating ESX/Med23 binding) comprise isoxazolidine based compounds (see, e.g., U.S. Pat. No. 7,786,310). In some embodiments, the isoxazolidine compounds comprise a functional group configured to mimic the amino acid portion of ESX known to bind with Med23 (e.g., at least a portion of amino acid residues 137-146 (SWIIELLE) (SEQ ID NO:1) within ESX) (e.g., thereby facilitating binding of the isoxazolidine compound within the respective region).

For example, in some embodiments, isoxazolidine compounds having the following formula are provided:

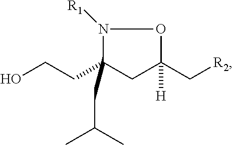

including salts, esters and prodrugs thereof. In some embodiments, R1 is a functional group configured to mimic the amino acid portion of ESX known to bind with Med23 (e.g., at least a portion of amino acid residues 137-146 (SWIIELLE) (SEQ ID NO:1) within ESX) (e.g., thereby facilitating binding of the isoxazolidine compound within the respective region). In some embodiments, R1 is a functional group configured to mimic the effect of Tryptophan 138 within ESX known to be essential for the specificity of the ESX-Med23 interaction (e.g., thereby facilitating binding of the isoxazolidine compound within the respective region). In some embodiments, R1 is a functional group configured to mimic the formation of a hydrophobic surface along an amphipathic helix within at least a portion of amino acids 137-146 of ESX that interacts with Med23 (e.g., thereby facilitating binding of the isoxazolidine compound within the respective region). In some embodiments, R1 is

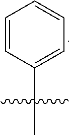 [SMILES: *c1ccccc1]

In some embodiments, R1 is

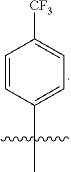 [SMILES: *c1ccc(C(F)(F)F)cc1]

In some embodiments, R1 is

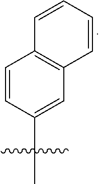 [SMILES: *c1ccc2ccccc2c1]

In some embodiments, R1 is

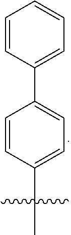 [SMILES: *c1ccc(-c2ccccc2)cc1]

The compounds are not limited to a particular structure.

In some embodiments, R2 is a functional group inhibiting binding to a DNA binding domain for a gene of interest. For example, in embodiments where the isoxazolidine compound is functioning as a competitive inhibitor for molecules that bind the Med23 binding region within ESX (e.g., amino acid residues 137-146 of ESX), R2 is any functional group that would inhibit binding with a DNA binding domain of a gene of interest. In such embodiments, R2 is N₃. In some embodiments, R2 is a DNA binding domain for any gene of interest (e.g., EGFR).

In some embodiments, the following small molecules that inhibit ESX-mediated transcription through interacting (e.g., binding) with at least a portion of the eight amino acid (137-SWIIELLE-146) (SEQ ID NO:1) α-helical region within ESX (e.g., the Med23/ESX interaction site within ESX) (e.g., thereby attenuating ESX/Med23 binding) are provided:

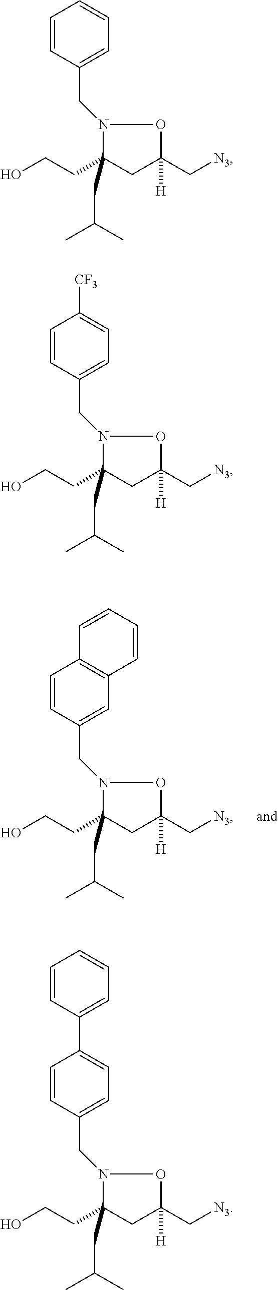

(biphenyl isoxazolidine) (see, e.g., Lee et al., 2009).

The present invention is not limited to the inhibition of a particular type of ESX-mediated transcription with small molecules.

In some embodiments, the small molecules inhibit ESX-mediated ErbB2(Her2) transcription (see, e.g., Chang et al., 1997; Lee et al., 2009). In some embodiments, the small molecules inhibit ESX-mediated ErbB2(Her2) transcription through attenuating ESX/Med23 binding.

Experiments conducted during the course of developing embodiments for the present invention demonstrated that EGFR transcription is regulated by ESX. As such, in some embodiments, the small molecules inhibit ESX-mediated EGFR transcription. The small molecules are not limited to a particular manner of inhibiting EGFR transcription. In some embodiments, EGFR transcription is inhibited through attenuating binding between ESX and Med23. In some embodiments, EGFR transcription is inhibited through the binding of small molecules that are configured to mimic at least a portion of the eight amino acid (137-SWIIELLE-146) (SEQ ID NO:1) α-helical region within ESX (e.g., the Med23/ESX interaction site within ESX) (e.g., thereby attenuating ESX/Med23 binding).

The foregoing ESX-mediated transcriptional inhibitors (e.g., small molecules capable of binding with at least a portion of the eight amino acid (137-SWIIELLE-146) (SEQ ID NO:1) α-helical region within ESX) (e.g., biphenyl isoxazolidine) can be present in pharmaceutical compositions comprising a compound described herein (e.g., biphenyl isoxazolidine) and a pharmaceutically acceptable carrier. In certain embodiments, the pharmaceutical composition further comprises a second therapeutic agent. In certain embodiments, the second therapeutic is directed toward inhibiting EGFR and/or Her2 expression, activity, and/or transcription. Examples of such therapeutics include, but are not limited to, monoclonal antibody inhibitors (e.g., cetuximab, panitumumab, zalutumubab, nimotuzumab, matuzumab), tyrosine kinase inhibitors (e.g., afatinib, gefitinib, erlotinib, lapatinib), and/or any therapeutic agents known/used for treating EGFR related disorders (e.g., cancer (e.g., HNSCC, lung cancer, colorectal cancer) (e.g., AP26113 (e.g., ARIAD Pharmaceuticals), potato carboxypeptidase inhibitors (see, e.g., Blanco-Aparicio, et al 1998).

### II. Methods for Identifying ESX-Mediated Transcription Modulators

The present invention provides methods for identifying small molecules capable of modulating (e.g., inhibiting, enhancing) ESX-mediated transcription. The methods are not limited to a particular type of ESX-mediated transcription. In some embodiments, the ESX-mediated transcription is EGFR transcription. In some embodiments, the ESX-mediated transcription is ErbB2(Her2) transcription.

The present invention is not limited to particular methods for identifying small molecules capable of modulating (e.g., inhibiting, enhancing) ESX-mediated transcription (e.g., EGFR transcription; ErbB2(Her2) transcription). In some embodiments, the methods include providing host cells expressing ESX and a gene whose transcription is regulated by ESX, ESX-mediated transcription coactivating compounds, and a potential ESX-mediated transcription modulator, delivering to the host cells an effective amount of the potential ESX-mediated transcription modulator, and detecting changes in ESX-mediated transcription (e.g., inhibited transcription, enhanced transcription).

For example, methods for detecting an ESX-mediated EGFR transcription inhibitor include, for example, providing host cells expressing ESX, Med23, and EGFR, and providing a potential ESX-mediated EGFR transcription inhibitor, delivering to the host cells an effective amount of the potential ESX-mediated EGFR transcription inhibitor, and detecting changes in EGFR transcription. Generally, a resulting attenuation in EGFR transcription indicates the potential ESX-mediated EGFR transcription inhibitor as a ESX-mediated EGFR transcription inhibitor. In some embodiments, additional comparisons can be made between potential ESX-mediated transcription inhibitors and known ESX-mediated EGFR transcription inhibitors (e.g., biphenyl isoxazolidine).

Similarly, in some embodiments, methods for detecting an ESX-mediated EGFR transcription enhancer include, for example, providing host cells expressing ESX, Med23, and EGFR, and providing a potential ESX-mediated EGFR transcription enhancer, delivering to the host cells an effective amount of the potential ESX-mediated EGFR transcription enhancer, and detecting changes in EGFR transcription. Generally, a resulting increase in EGFR transcription indicates the potential ESX-mediated EGFR transcription enhancer as a ESX-mediated EGFR transcription enhancer.

In some embodiments, algorithms such as TFSEARCH or JASPAR are used to identify putative ESX binding sites in a gene promoter of interest (e.g., EGFR promoter) (see, e.g., Example 1). Upon identification of such putative ESX binding sites in a gene promoter of interest, small molecules can be constructed as ESX-mediated transcription modulators (e.g., ESX-mediated transcription inhibitors, ESX-mediated transcription enhancers).

### III. Methods for Regulating ESX-Mediated Gene Expression

The present invention provides methods for regulating expression of a gene known to be regulated by ESX. For example, the present invention provides methods for regulating expression of EGFR comprising providing, for example, host cells (e.g., in vivo, ex vivo, in vitro) (e.g., HNSCC cells) expressing ESX, Med23, and EGFR, and ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine), and delivering to the host cells an effective amount of the ESX-mediated transcription inhibitors such that EGFR expression is modified (e.g., EGFR expression is attenuated). In some embodiments, EGFR expression is suppressed. In some embodiments, resulting cell proliferation, cell invasion, and/or cell migration can further be regulated/monitored as a result of such gene expression regulation.

### IV. ESX-Mediated Transcription Based Therapeutics and Research Applications

The present invention provides methods for regulating ESX-mediated transcription of a gene of interest in a subject for the purpose of, for example, analyzing the effect of a ESX-mediated transcription modulator, modulating transcription to assist with therapy (e.g., co-administered with existing therapies) or as a standalone therapy, comprising: providing a subject and a ESX-mediated transcription modulator and delivering to the subject an effective amount of the ESX-mediated transcription modulator such that expression of the gene of interest is modified (e.g., inhibited, enhanced).

Experiments conducted during the course of developing embodiments for the present invention determined that ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) provide therapeutic benefits to subjects (e.g., human patients) suffering from various disorders having aberrant EGFR expression. In particular, it was shown that ESX is elevated and associated with EGFR and Her2 in HNSCC, that targeting ESX is sufficient to dampen the oncogenic phenotype for HNSCC, that biphenyl isoxazolidine effectively suppresses ESX transcriptional activity leading to a decrease in EGFR and Her2 levels and inhibition of cell invasion, motility, and proliferation. Examples of disorders having aberrant EGFR expression include, but are not limited to, cancer (e.g., HNSCC, lung cancer (non-small cell lung carcinoma), colorectal cancer, breast cancer).

In one aspect, the invention provides a method of treating diseased cells, tissues, organs, or pathological conditions and/or disease states associated with EGFR expression (e.g., an EGFR related disorder (e.g., HNSCC, lung cancer (non-small cell lung carcinoma), colorectal cancer, breast cancer)), comprising administering a therapeutically effective amount of an ESX-mediated transcription inhibitor (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) to a subject in need thereof to ameliorate a symptom of the condition. In certain embodiments, the subject is an animal (e.g., a mammalian patient including, but not limited to, humans and veterinary animals).

Experiments conducted during the course of developing embodiments for the present invention further demonstrated that targeting EGFR/Her2 levels and kinase activities simultaneously is a highly efficacious strategy to ablate the proliferation of HNSCC cells. In particular, experiments conducted during the course of developing embodiments for the present invention demonstrated that biphenyl isoxazolidine enhanced the anti-proliferative effects of afatinib, gefitinib and lapatinib, three EGFR tyrosine kinase inhibitors. Indeed, the experiments indicate inhibition of ESX to reduce EGFR and Her2 levels as an approach to enhance the response rate of HNSCC patients to current anti-EGFR therapeutics. Accordingly, in certain other embodiments, the therapeutic methods further comprise co-administering to the subject a therapeutic agent selected from the group consisting of an EGFR monoclonal antibody inhibitor (e.g., cetuximab, panitumumab, zalutumubab, nimotuzumab, matuzumab), a tyrosine kinase inhibitor (e.g., afatinib, gefitinib, erlotinib, lapatinib), and/or any therapeutic agents known/used for treating EGFR related disorders (e.g., cancer (e.g., HNSCC, lung cancer, colorectal cancer) (e.g., AP26113 (e.g., ARIAD Pharmaceuticals), potato carboxypeptidase inhibitors (see, e.g., Blanco-Aparicio, et al 1998).

Generally, it is contemplated that the ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) be co-administered with any anti-cancer agent (e.g., Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N-(trans-4-methylcyclohexyl-N--nitrosourea (MeCCNU); N-(2-chloroethyl)-N-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen 1 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex. One particularly preferred class of anticancer agents are taxanes (e.g., paclitaxel and docetaxel). Another important category of anticancer agent is annonaceous acetogenin.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, methods provided herein comprise administering one or more ESX-mediated transcription inhibitors with radiation therapy. The methods provided herein are not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

Experiments conducted during the course of developing embodiments for the present invention demonstrated that combinations of transcriptional inhibitors of erbB2 and existing therapeutic agents that target erbB2 activity and lifetime lead to a synergistic increase in activity, with dose reductions as high as 30 fold compared to individual agents (see Example 5). Accordingly, in certain embodiments, the present invention provides methods for treating a subject having a disorder having elevated erbB2 expression. The present invention is not limited to particular methods for treating disorders having elevated erbB2 expression. In some embodiments the methods involve, for example, co-administering to the subject an effective amount of an ESX-mediated transcription inhibitor, and one or more agents known to target the activity and lifetime of the erbB2 oncoprotein. Any type of subject is contemplated for such methods (e.g., human, dog, cat, cow, ape, etc.).

The methods are not limited to a particular disorder having elevated erbB2 expression. In some embodiments, the disorder is a cancer or cancer related disorder (e.g., breast cancer, stomach cancer, ovarian cancer, endometrial carcinoma (see, e.g, Santin, 2008). In some embodiments, the cancer is breast cancer.

The methods are not limited to a particular type of ESX-mediated transcription inhibitor. In some embodiments, the ESX-mediated transcription inhibitor is an isoxazolidine compound as described herein (e.g., biphenyl isoxazolidine).

The methods are not limited to particular agents known to target the activity and lifetime of the erbB2 oncoprotein. In some embodiments, the agent is a tyrosine kinase inhibitor (e.g., afatinib, gefitinib, erlotinib, lapatinib (see Example 5). In some embodiments, the agent is an anti-tumor antibiotic (e.g., benzoquinone ansamycin antibiotics (e.g., geldanamycin (see, e.g., Bedin, 2004) (see Example 5), 17-N-Allylamino-17-demethoxygeldanamycin (17-AAG), 17-Dimethylaminoethylamino-17-demethoxygeldanamycin (17-DMAG))).

### V. Pharmaceutical Compositions, Formulations, and Exemplary Administration Routes and Dosing Considerations

Exemplary embodiments of various contemplated medicaments and pharmaceutical compositions are provided below.

### A. Preparing Pharmaceutical Formulations

The ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) are useful in the preparation of pharmaceutical formulation, also synonymously referred to herein as “medicaments,” to treat a variety of conditions associated with EGFR expression (e.g., cancer (e.g., HNSCC, lung cancer (non-small cell lung carcinoma), colorectal cancer, breast cancer)). The methods and techniques for preparing medicaments of ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) are well-known in the art. Exemplary pharmaceutical formulations and routes of delivery are described below.

One of skill in the art will appreciate that any one or more of the ESX-mediated transcription inhibitors described herein are prepared by applying standard pharmaceutical manufacturing procedures. Such pharmaceutical formulations can be delivered to the subject by using delivery methods that are well-known in the pharmaceutical arts.

### B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate. Likewise, those for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-close or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent. It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

In some embodiments, the compounds of the present invention are provided in unsolvated form or are in non-aqueous solutions (e.g., ethanol). The compounds may be generated to allow such formulations through the production of specific crystalline polymorphs compatible with the formulations.

In certain embodiments, the present invention provides instructions for administering said compound to a subject. In certain embodiments, the present invention provides instructions for using the compositions contained in a kit for the treatment of conditions characterized by the dysregulation of apoptotic processes in a cell or tissue (e.g., providing dosing, route of administration, decision trees for treating physicians for correlating patient-specific characteristics with therapeutic courses of action). In certain embodiments, the present invention provides instructions for using the compositions contained in the kit to treat a variety of medical conditions associated with irregular EGFR expression (e.g., HNSCC).

### C. Exemplary Administration Routes and Dosing Considerations

Various delivery systems are known and can be used to administer therapeutic agents (e.g., exemplary compounds as described in Section II above) of the present invention, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

It is contemplated that the agents identified can be administered to subjects or individuals susceptible to or at risk of developing pathological growth of target cells and correlated conditions. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or non-aqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

### D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine). Indeed, experiments conducted during the course of developing embodiments for the present invention demonstrated that targeting EGFR/Her2 levels and kinase activities simultaneously is a highly efficacious strategy to ablate the proliferation of HNSCC cells. Accordingly, in certain other embodiments, the therapeutic methods further comprise co-administering to the subject a therapeutic agent selected from the group consisting of an EGFR monoclonal antibody inhibitor (e.g., cetuximab, panitumumab, zalutumubab, nimotuzumab, matuzumab), a tyrosine kinase inhibitor (e.g., afatinib, gefitinib, erlotinib, lapatinib), and/or any therapeutic agents known/used for treating EGFR related disorders (e.g., cancer (e.g., HNSCC, lung cancer, colorectal cancer) (e.g., AP26113 (e.g., ARIAD Pharmaceuticals), potato carboxypeptidase inhibitors (see, e.g., Blanco-Aparicio, et al 1998). Indeed, any type of anti-cancer agent may be co-administered with a ESX-mediated transcription inhibitor for purposes of treating a disorder having aberrant EGFR expression.

In co-administration procedures, the agents may be administered concurrently or sequentially. In one embodiment, the ESX-mediated transcription inhibitors (e.g., small molecules capable of binding with at least a portion of the eight amino acid (137-SWIIELLE-146) (SEQ ID NO:1) α-helical region within ESX) (e.g., the Med23/ESX interaction site within ESX) (e.g., biphenyl isoxazolidine) are administered prior to the other active agent(s). The pharmaceutical formulations and modes of administration may be any of those described above. In addition, the two or more co-administered chemical agents, biological agents or radiation may each be administered using different modes or different formulations.

The additional agents to be co-administered can be any of the well-known agents in the art for a particular disorder, including, but not limited to, those that are currently in clinical use and/or experimental use.

### VI. Other Embodiments

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The invention, now being generally described, will be more readily understood by reference to the following examples, which are included merely for purposes of illustration of certain aspects and embodiments of the present invention, and are not intended to limit the invention.

### Example 1

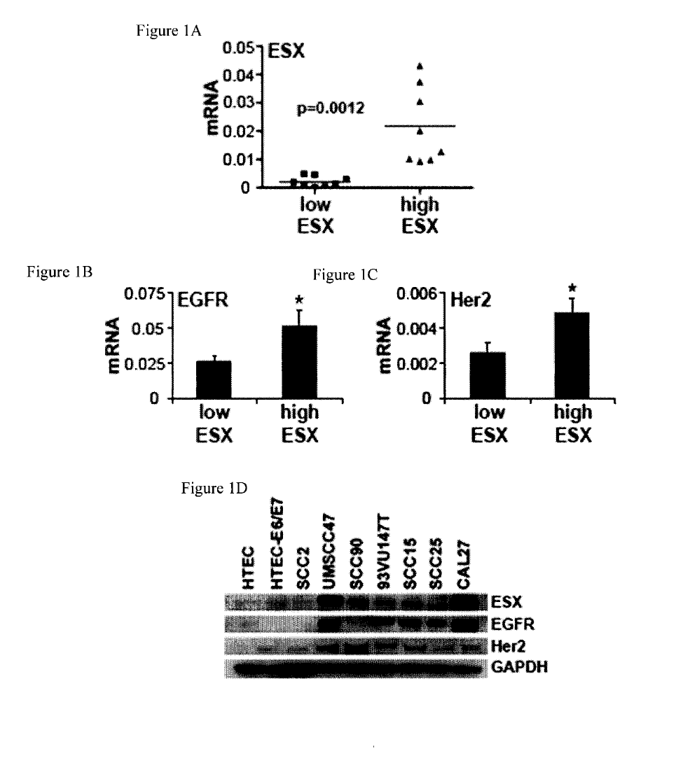

This example shows that ESX is elevated and associated with EGFR and Her2 in HNSCC. In silico analysis using TFSEARCH identified multiple putative ESX binding sites in the EGFR promoter suggesting that ESX may directly regulate EGFR expression. To begin to examine the role of ESX in the regulation of EGFR family members in HNSCC, ESX, EGFR, and Her2 expression in primary tumors from previously untreated HNSCC patients was determined (FIG. 1). There was considerable range (0.00007 to 0.04310) in ESX mRNA expression in primary HNSCC tumors (n=16). ESX expression was stratified into two groups; low and high ESX. Patients with the highest 8 ESX expression were binned into the high ESX group and patients with the lowest 8 ESX expression were binned into the low ESX group. ESX expression was 0.022±0.005 for the high ESX group and 0.002±0.001 for the low ESX group (FIG. 1A). An 11-fold increase in ESX expression was found between the high and low ESX HNSCC patients (p=0.0012). In comparison to the low ESX patients, the high ESX patients had a 93% (p<0.05) increase in EGFR expression and an 86% (p<0.04) increase in Her2 expression (FIGS. 1B and 1C). EGFR expression was about 10-fold higher than Her2 expression in these clinical samples consistent with the published literature reporting EGFR as the predominant EGFR family member that is expressed in HNSCC. Pearson's analyses demonstrated a significant correlation between ESX and EGFR (p=0.029) and ESX and Her2 (p=0.004). Similar to primary HNSCC tumor data, HNSCC cell lines had differential levels of ESX (FIG. 1D). ESX levels were higher in the panel of HNSCC cell lines compared to human primary tonsillar epithelial cells (HTEC) with the exception of SCC2. These results indicate that ESX is elevated and associated with EGFR and Her2 in HNSCC.

### Example 2

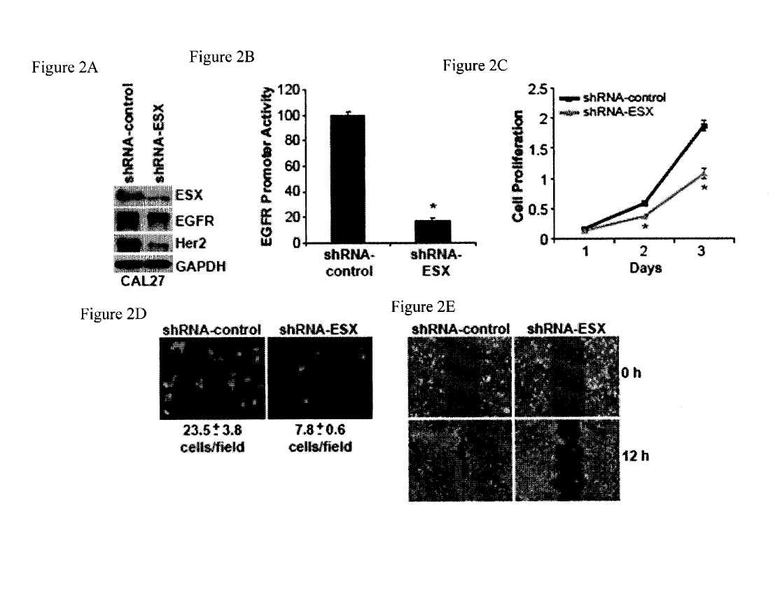

This example shows that targeting ESX is sufficient to dampen the oncogenic phenotype indicating ESX as a novel druggable target for HNSCC. It is clear that EGFR is almost universally increased in HNSCC, however, in contrast to other carcinomas, EGFR amplification is low in this patient population. EGFR amplification was reported to range between 10-15% in HNSCC (see, e.g., Chung et al., 2006; Licitra et al.; Temam et al., 2007). Thus, the major molecular mechanism involved in EGFR over-expression in HNSCC remained to be elucidated. Results using primary HNSCC tumors revealed a significant association between ESX and EGFR. In addition, analysis of the EGFR promoter identified multiple putative ESX binding sites. These observations indicates that ESX directly hyperactivates the EGFR promoter to drive EGFR expression. As shown in FIG. 2A, CAL27/shRNAESX cells showed a dramatic decrease in ESX, EGFR and Her2 levels compared to CAL27/shRNA-control cells. Genetic knockdown of ESX resulted in an 83±2% inhibition (p<0.001, n=9) in EGFR promoter activity in CAL27 cells (FIG. 2B). These results show that ESX regulates EGFR through increased activation of the EGFR promoter and thus, identified a novel molecular mechanism for EGFR over-expression in HNSCC. Furthermore, CAL27/shRNA-ESX cells showed a significant decrease in cell proliferation (15±2% inhibition, p<0.005, n=3), cell invasion (67±3% inhibition, p<0.005, n=6), and cell migration compared to CAL27/shRNA-control cells. These observations indicate that targeting ESX is sufficient to dampen the oncogenic phenotype suggesting that ESX may be a novel druggable target for HNSCC.

### Example 3

This example shows that biphenyl isoxazolidine can effectively suppress ESX transcriptional activity leading to a decrease in EGFR and Her2 levels and inhibition of cell invasion, motility, and proliferation.

ESX interacts with multiple coactivator proteins to regulate gene transcription. Med23, the most well characterized ESX coactivator, interacts with ESX to regulate Her2 transcription. An eight amino acid (137-SWIIELLE-146) (SEQ ID NO:1) α-helical region in ESX was reported to mediate the interaction between ESX and Med23 (see, e.g., Asada et al., 2002). Tryptophan 138 was shown to be essential for the specificity of the ESX-Med23 interaction (see, e.g., Asada et al., 2002). In addition, NMR spectroscopy suggests that W138 along with 1139, 1140, L142, and L143 form a hydrophobic surface along an amphipathic helix that interacts with Med23 (see, e.g., Asada et al., 2002). The binding interaction between ESX and Med23 was shown to be disrupted with a small molecule α-helix mimic of ESX, wrenchnolol (Shimogawa et al., 2004). Wrenchnolol decreased Her2 expression and inhibited cell proliferation of SKBR3 Her2-positive breast carcinoma cells (see, e.g., Shimogawa et al., 2004). Recently, a novel α-helix ESX mimic, biphenyl isoxazolidine, was designed and synthesized to block the interaction between ESX-Med23 (see, e.g., Lee et al., 2009). Similar to wrenchnolol, biphenyl isoxazolidine decreased Her2 expression and inhibited cell proliferation of SKBR3 cells (see, e.g., Lee et al., 2009). These two studies showed that targeting the ESX-Med23 interaction is feasible and moreover, demonstrated that inhibition of transcription factor activation with small molecules is a novel and promising avenue for anti-cancer drug development.

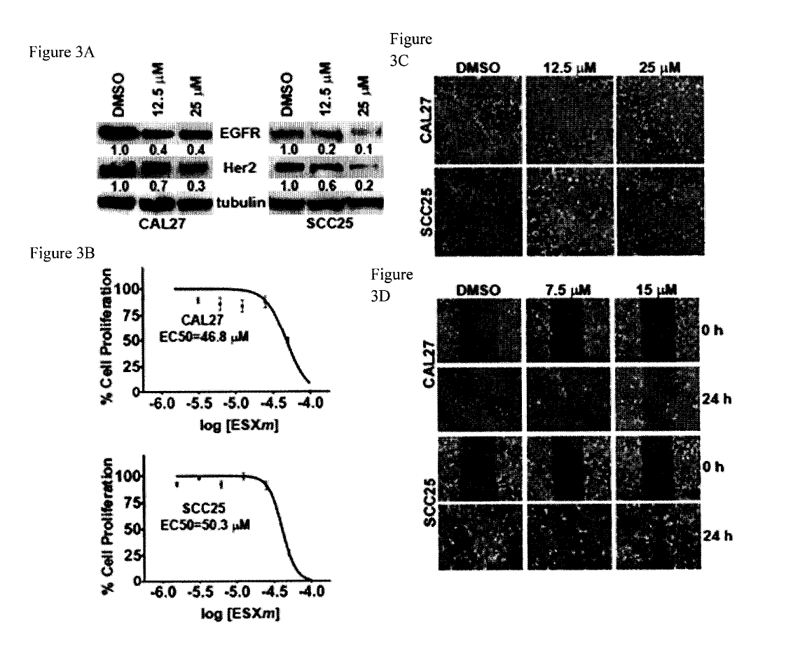

To assess if ESX is a druggable target in HNSCC, the effects of biphenyl isoxazolidine was examined in CAL27 and SCC25 cells, two HNSCC cell lines with high endogenous ESX (FIG. 3). A dose-dependent decrease in EGFR and Her2 levels in CAL27 and SCC25 cells was observed in response to biphenyl isoxazolidine. Cell proliferation was inhibited with biphenyl isoxazolidine exposure. The IC50 for cell proliferation at 24 hours of treatment was 46.8 μmol/L for CAL27 and 50.3 μmol/L for SCC25. Cell invasion and migration were dramatically suppressed in CAL27 and SCC25 cells with 12.5 μmol/L and 15 μmol/L biphenyl isoxazolidine, respectively. The concentration of biphenyl isoxazolidine needed to inhibit cell invasion and cell migration is much lower than required to inhibit cell proliferation. This observation argues that the EGFR gene dosage threshold to modulate cell invasion, migration, and proliferation is different; cell migration and invasion requires a higher level of EGFR than cell proliferation. Moreover, these results demonstrate that inhibition of cell invasion and migration mediated by biphenyl isoxazolidine is not due, for example, to a general decrease in cell viability but due, for example, to dampening of signal transduction pathways specific to cell morphology and movement. EGFR and Her2 are well recognized as regulators of cell invasion, migration, and proliferation. Taken together, these results show that biphenyl isoxazolidine can effectively suppress ESX transcriptional activity leading to a decrease in EGFR and Her2 levels and inhibition of cell invasion, motility, and proliferation.

### Example 4

This example demonstrates that targeting EGFR/Her2 levels and kinase activities simultaneously is a highly efficacious strategy to ablate the proliferation of HNSCC cells.

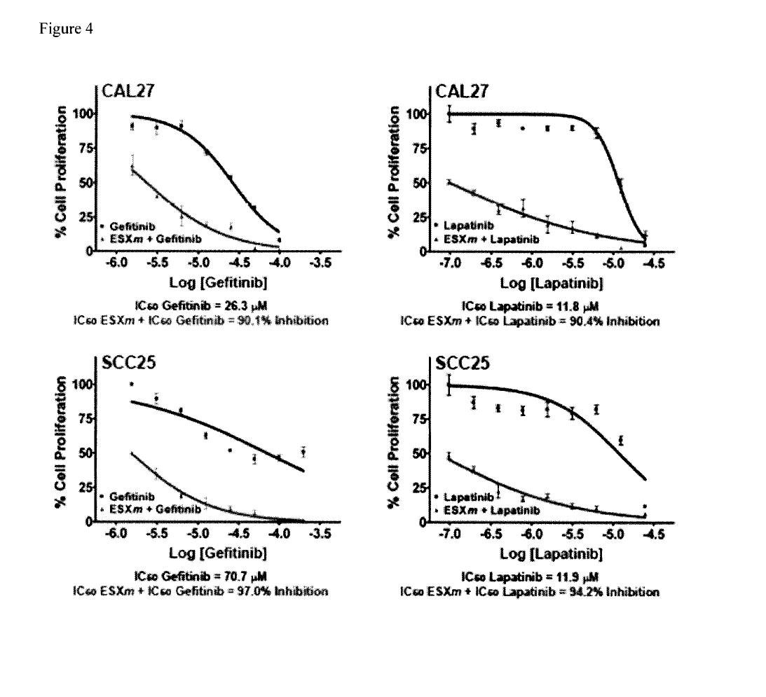

Monotherapy with EGFR inhibitors, such as cetuximab, a humanized anti-EGFR antibody, or TKIs, such as gefitinib, lapatinib, and erlotinib, has yielded very modest activity in HNSCC patients to date. These clinical observations provide evidence that targeting EGFR kinase dependent activity and/or downstream signaling may not be robust enough to reach the maximal anti-cancer therapeutic response. A combination of two different yet complementary approaches to target the EGFR family members, reduction of EGFR/Her2 levels with biphenyl isoxazolidine and suppression of EGFR/Her2 kinase activity with TKIs, may result in greater therapeutic efficacy. To test this hypothesis, CAL27 and SCC25 cells were treated with gefitinib, an EGFR TKI, or lapatinib, a dual EGFR/Her2 TKI, at various concentrations with and without an IC50 dose of biphenyl isoxazolidine (FIG. 4). Single agent gefitinib and lapatinib inhibited the proliferation of CAL27 and SCC25 cells after 24 hours of treatment. The IC50 was 26.3 μmol/L for gefitinib and 11.8 μmol/L for lapatinib in CAL27 cells. SCC25 cells were highly resistant to gefitinib but sensitive to lapatinib; IC50 was 70.7 μmol/L for gefitinib and 11.9 μmol/L for lapatinib Importantly, the combination treatment with IC50 biphenyl isoxazolidine and IC50 gefitinib or lapatinib decreased cell proliferation by greater than 90% in CAL27 and SCC25 cells. These results demonstrate that targeting EGFR/Her2 levels and kinase activities simultaneously is a highly efficacious strategy to ablate the proliferation of HNSCC cells.

### Example 5

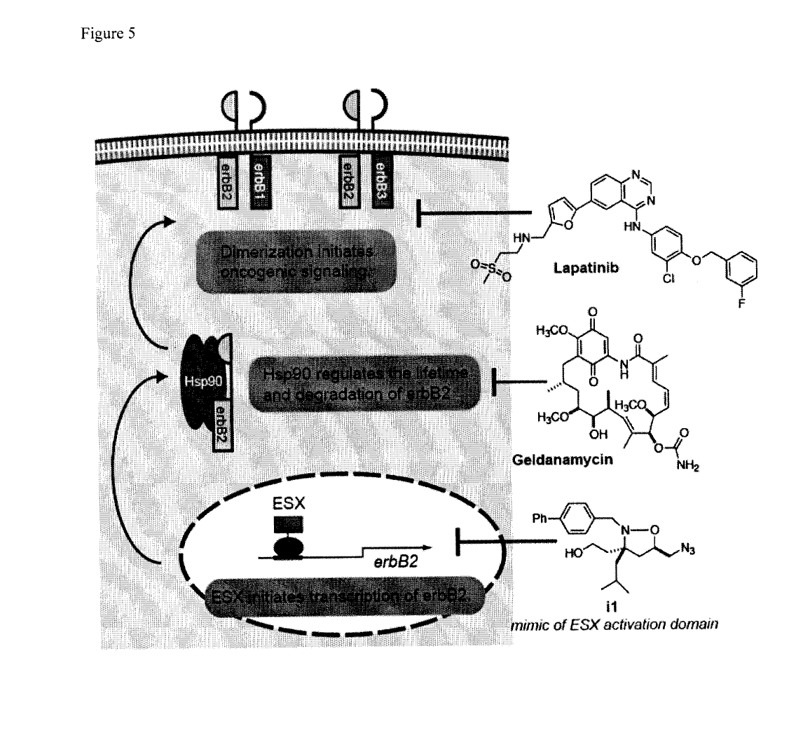

The erbB2 protein is a trans-membrane tyrosine kinase that is over expressed in approximately one quarter of breast cancers (see, e.g., Slamon, et al., 1989), where it has been shown to drive an aggressive phenotype marked by more rapid metastasis and shorter life expectancy than breast cancers that do not over-express erbB2 (see, e.g., Yarden, 2000; Slamon, 1987). Furthermore, erbB2 over-expressing (erbB2+) cancer cells are known to undergo growth arrest and cell death if erbB2 expression is suppressed (see, e.g., Menendez, 2004). The clinical significance of erbB2 overexpression can be seen in the variety of existing treatments designed to suppress erbB2 signaling, including antibodies that target the protein's extracellular domain (see, e.g., Nahta, 2007) and tyrosine kinase inhibitors which target the protein's ability trans-phosphorylate other members of the erbB family and initiate cell survival and proliferation programs (FIG. 5) (see, e.g., Xia, 2005). These approaches have met with difficulty in clinical practice, but an increasing body of evidence suggests that although erbB2 driven cancers are adept at compensating for partial inhibition of erbB2 activity, they are still vulnerable to interventions that reduce erbB2 levels (see, e.g., Kong, 2008; Sergina, 2007).

One point of intervention along the erbB2 pathway is Hsp90, part of a chaperone complex that maintains erbB2 stability and assists in membrane localization (see, e.g., Roe, 1999; Isaacs, 2003). The natural product geldanamycin reduces cellular erbB2 levels by binding to Hsp90 and inhibiting its function (FIG. 5) but its toxicity prevents its use as a therapeutic agent (see, e.g., Roe, 1999; Isaacs, 2003).

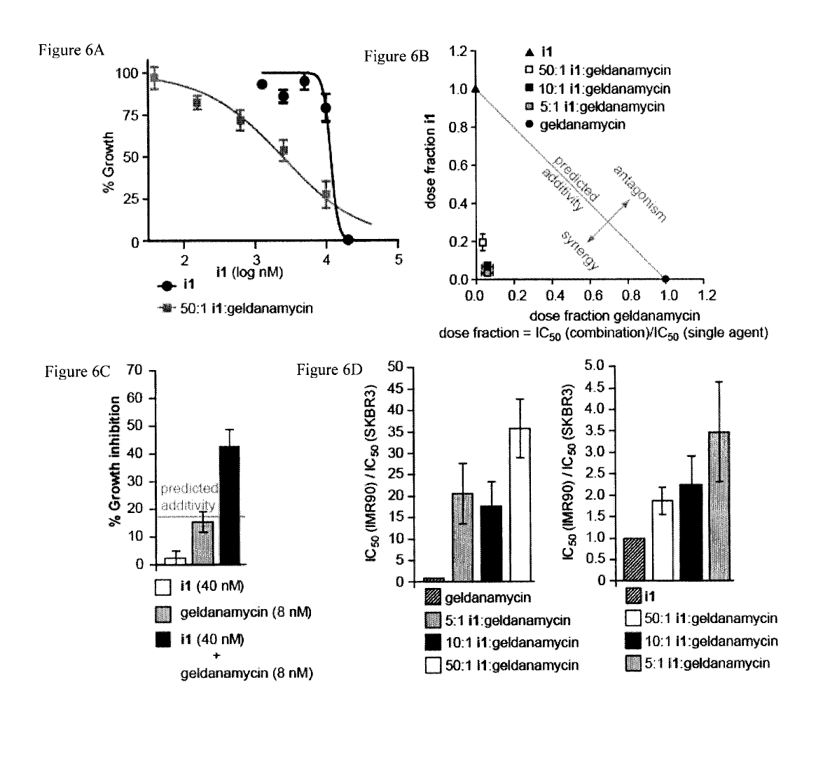

Thus experiments were conducted to test the hypothesis that dual targeting of the erbB2 pathway with the two protein-protein interaction inhibitors geldanamycin and biphenyl isoxazoline would synergistically increase potency and specificity relative to the individual agents. As shown in FIG. 6a, a 50:1 combination of biphenyl isoxazoline:geldanamycin resulted in an IC50 in SkBr3 (erbB2+) cells that is >10-fold lower than biphenyl isoxazoline alone. To test if the potency increase is truly synergistic, both the isobologram and multiplicative additivity (Bliss) models were employed. For the former, the IC50s of fixed ratios of biphenyl isoxazoline:geldanamycin were measured and compared to a hypothetical case representing additivity, in which both components act as though they are the same agent (FIG. 6b) (see, e.g., Berenbaum, 1989). IC50 ratios (combination:single agent) that fall below the additivity line are indicative of positive synergy and by this measure, the combinations of the two PPI inhibitors exhibit an impressive degree of synergy. The most efficacious biphenyl isoxazoline:geldanamycin combination was 5:1, and this combination is also synergistic as defined by the multiplicative additivity or Bliss model (FIG. 6c) (see, e.g., Berenbaum, 1989; Borisy, 2003). This degree of synergy increased proliferation from combination treatment (Supporting Figure S7b-c). In addition, the combination of geldanamycin and biphenyl isoxazoline concomitantly produced an 85% drop in erbB2 levels

### (Supporting Figure S7a).

As outlined earlier, biphenyl isoxazoline displays modest selectivity for erbB2+ cancer cell lines and geldanamycin is broadly toxic. However, combinations of biphenyl isoxazoline and geldanamycin show increased selectivity for erbB2+ cancer cells when compared to non-tumorigenic IMR90 cells, cells whose growth is not driven by erbB2 (FIG. 6d). This is most notable in comparison with geldanamycin alone, where the combinations produce a 20- to 35-fold selectivity improvement. These results indicate that the synergy is erbB2-dependent and not a result of general toxicity. These data further suggest that transcriptional inhibitors can be used in combinations with agents that have broad activity to selectively effect specific shared targets.

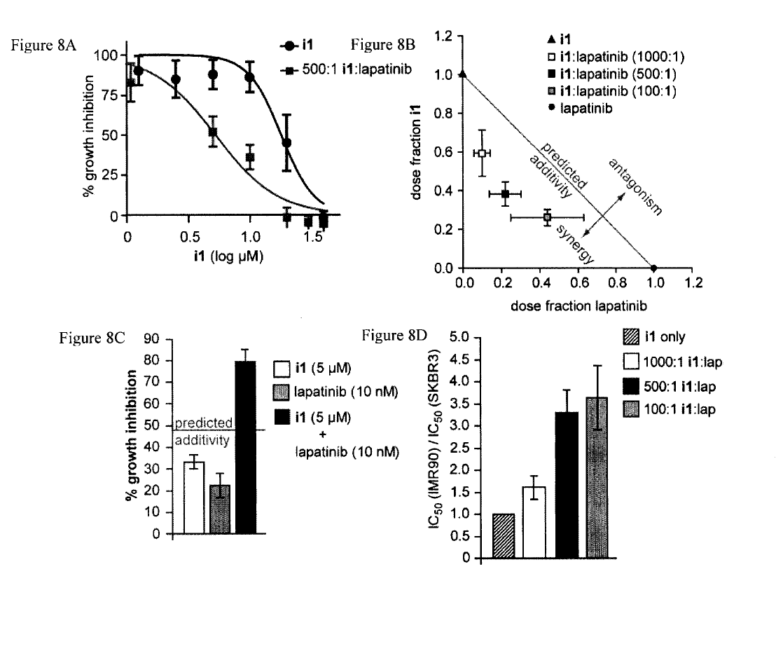

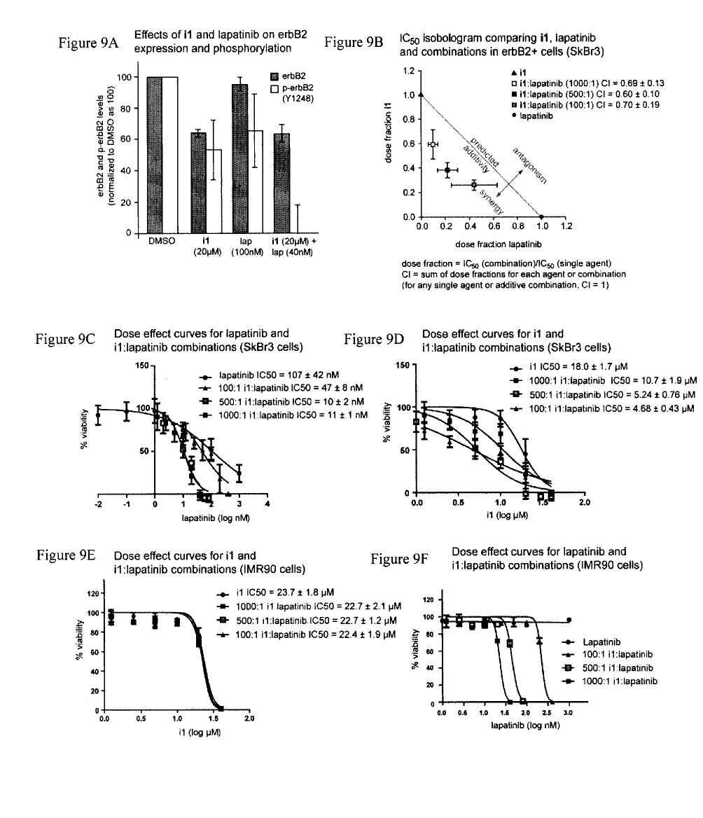

The potential for synergy between biphenyl isoxazoline and lapatinib, a reversible erbB2/erbB 1 kinase inhibitor that is used clinically in the treatment of erbB2+ cancers was next examined (FIG. 1) (see, e.g., Petrov, 2006). An initial trial of a 500:1 ratio of biphenyl isoxazoline:lapatanib produced a>10-fold decrease in the IC50 relative to biphenyl isoxazoline alone in SkBr3 (FIG. 8a). That this decrease was due to synergy was tested as before with via both the isobologram and multiplicative additivity (Bliss) methods in SkBr3 cells (FIG. 8b). The IC50 ratios of the over longer growth times, indicating robust inhibition of biphenyl isoxazoline:lapatinib combinations fell significantly below the additivity line, demonstrating a synergistic effect. Consistent with the impact on viability, biphenyl isoxazoline and lapatinib had moderate effects on erbB2 and phosphorylated erbB2 levels as single agents. However the biphenyl isoxazoline:lapatinib combination was significantly (p<0.05) more effective at reducing the total amount of active (phosphorylated) erbB2 than equivalent amounts of either biphenyl isoxazoline or lapatinib (FIG. 9a).

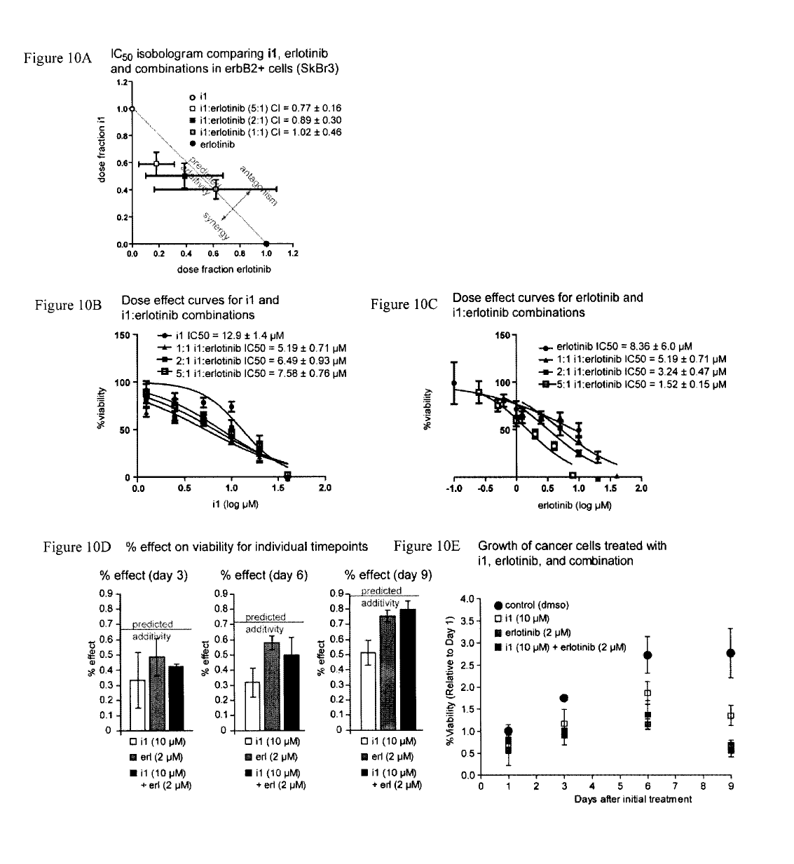

In addition to the increased potency, combinations of biphenyl isoxazoline and lapatinib are less toxic to erbB2-negative, non-tumorigenic IMR90 cells, leading to greater selectivity than the use of biphenyl isoxazoline in isolation (FIG. 8d). As an additional readout for synergy, colonies of SkBr3 cells were dosed with compound (5 μM biphenyl isoxazoline, 10 nM lapatinib or a combination of the two) for 9 days. The combination treatment was much more effective than either biphenyl isoxazoline or lapatinib in isolation or the multiplicative sum of the individual effects. In contrast, combinations of biphenyl isoxazoline and the erbB1 selective kinase inhibitor erlotinib29 did not display significant synergy (FIG. 10). This result is consistent with models that implicate the erbB2/erbB3 dimer as the primary driver of oncogenesis (see, e.g., Kong, 2008; Sergina, 2007).

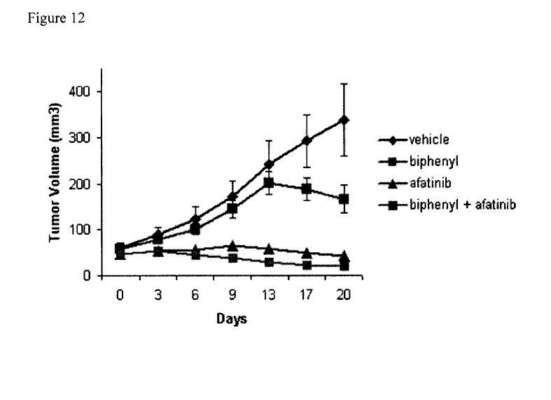

The anti-tumor efficacy of biphenyl isoxazolidine as single agent and in combination with afatinib, an irreversible EGFR/Her2 tyrosine kinase inhibitor, was assessed in a xenograft model of HNSCC. CAL27 HNSCC cells (1×10⁶) were implanted into the flank of 8-week-old athymic nude mice and tumors were allowed to develop without treatment. At 3 weeks post-tumor cell implantation, mice with established tumors were randomly assigned to four treatment arms; vehicle, biphenyl isoxazolidine (100 ng; 5× week; intratumoral injection), afatinib (20 mg/kg; 5× week; oral gavage), or biphenyl isoxazolidine and afatinib (see FIG. 12). As shown in FIG. 12, single-agent biphenyl isoxazolidine inhibited tumor growth by 51% (n=10, p<0.05) and single-agent afatinib suppressed tumor growth by 87% (n=10, p<0.01). The combination of biphenyl isoxazolidine and afatinib was the most active and blocked tumor growth by 94% (n=10. p<0.01). It should be noted that the mean tumor volume was 2.1-fold higher for the single-agent afatinib arm compared to the combination treatment arm (43 mm³ vs. 20 mm³) Importantly, the anti-tumor efficacy of the combination treatment arm was statistically superior to either single-agent biphenyl isoxazolidine (p<0.01) or single-agent afatinib (p<0.01). These results demonstrate that biphenyl isoxazolidine potentiates the anti-tumor efficacy of EGFR/Her2 TKIs in HNSCC.

### Example 6

This example provides materials and methods.

Materials: Isoxazolidine it was prepared as described previously (see, e.g., Lee, 2009). Lapatinib ditosylate and erlotinib were purchased from AK scientific, and geldanamycin was a generous gift. The identity and purity of all compounds were verified by NMR analysis and HPLC. Antibodies were purchased from Santa Cruz Biotechnology. Absorbance data was collected on a Tecan GENios Pro.

Calculation of synergy (see, e.g., Berenbaum, 1989; Borisy, 2003): IC₅₀s were calculated in Graphpad Prism v 5.0. All other calculations were performed in excel. Isobolograms were generated by computing dose fractions directly from the IC₅₀s. Dose fraction is defined as the dose of one component in a combination required to exert a given effect (usually IC₅₀, as in this case) divided by the dose of that component in isolation required to exert the same effect. Thus each combination reported has two dose fraction measurements (eg. dose fraction it and dose fraction lapatinib) that define the combinations x/y coordinates on the isobologram.

Dose fraction of A for combination AB=IC₅₀(A in AB)/IC₅₀(A in isolation)

Dose fraction of B for combination AB=IC₅₀(B in AB)/IC₅₀(B in isolation)

The sum of these x/y coordinates are the combination index. By definition, the CI for either agent in isolation is 1. For the null hypothesis (in which both agents act as though they are equivalent doses of the same agent) the CI will be 1. For combinations where the CI<1, synergy is present.

CI(combination index)=dose fraction A+dose fraction B.

Bliss additivity for a given combination was calculated by multiplying the fractional effect of the two components, according to the formula given below for combination AB whose components have effects eA and eB (which are expressed as fractional effects between 0 and 1) in isolation.

Predicted effect of AB=(eA+eB)−(eA*eB)

Mammalian Cell Culture: SkBr3 and IMR90 cells were purchase from ATCC and cultured in RPMI 1640 (SkBr3) or DMEM (IMR90) with 10% FBS and no antibiotics. For the experiments used to generate dose-effect curves for the isobolograms and selectivity experiments, cells were plated at 3000 cells per well in 96 well plates. After adhering overnight, media was changed to 2.5% FBS and compound (as a solution in DMSO) was added. New media and compound were added for each additional day of treatment (2 days in the case of lapatinib and 3 days in the case of geldanamycin). The day after the final treatment, cell viability was measured using WST-1 reagent (Roche) in accordance with the manufacturer's instructions. For the 9 day cell growth assays cells were plated at 15000 cells per well in 24 well plates (one for each timepoint to minimize potential for contamination). Doses at or below the IC50's from the above experiments were chosen to maximize the dynamic range of the assay. These experiments were otherwise run in the same way as those done in 96 well plates.

ErbB2 and p-erbB2 ELISA: ELISA assays were performed based on those published elsewhere._{[4]} In brief, SkBr3 cells were plated in 10% FBS at 15000 cells per well in 24 well plates. After adhering overnight, media was changed to 2.5% FBS and compound was added. After 24 hours, media was removed carefully and cells were fixed and permeabilized with cold (−20° C.) methanol. The cells were then washed twice with TBST, blocked for 2 hours at room temperature with superblock (TBST solution from Pierce), incubated with the primary antibody overnight at 4° C. (as a 1:500 solution in superblock). Cells were then washed twice more with TBST and incubate with secondary antibody for 2 hours at room temperature (as a 1:1000 solution in superblock). After being washed 3 times with TBST, Slow TMB (Pierce) was added to measure antibody levels in accordance with the manufacturer's instructions and the absorbance at 370 nm was measured. The cells were washed 3 more times with TBST and total protein levels were measured at 560 nm using BCA reagent (Pierce). ErbB2 and p-erbB2 levels were normalized to total protein concentration by calculating (A370-A370blank)/(A560-A560 blank), where the A370 blank was from TMB treated cells not treated with a primary antibody, and the A560 blank was BCA reagent in wells that did not have cells plated in them. The resulting ratios were then normalized to cells treated with DMSO.

### REFERENCES

Asada S, Choi Y, Yamada M, Wang S C, Hung M C, Qin J et al (2002). External control of Her2 expression and cancer cell growth by targeting a Ras-linked coactivator. Proc Natl Acad Sci USA 99: 12747-52; incorporate herein by reference in its entirety; incorporate herein by reference in its entirety.
Bedin M, Gaben A M, Saucier C, Mester J. Int J Cancer. 2004 May 1;109(5):643-52. Geldanamycin, an inhibitor of the chaperone activity of HSP90, induces MAPK-independent cell cycle arrest; herein incorporated by reference in its entirety.
Blanco-Aparicio C, Molina M A, Fernández-Salas E, Frazier M L, Mas J M, Querol E, Avilés F X, de Llorens R (May 1998). “Potato carboxypeptidase inhibitor, a T-knot protein, is an epidermal growth factor antagonist that inhibits tumor cell growth”. J. Biol. Chem. 273 (20): 12370-7; herein incorporated by reference in its entirety.
Cabral A, Fischer D F, Vermeij W P, Backendorf C (2003). Distinct functional interactions of human Skn-1 isoforms with Ese-1 during keratinocyte terminal differentiation. J Biol Chem 278: 17792-9; incorporate herein by reference in its entirety; herein incorporated by reference in its entirety.
Chang C H, Scott G K, Kuo W L, Xiong X, Suzdaltseva Y, Park J W et al (1997). ESX: a structurally unique Ets overexpressed early during human breast tumorigenesis. Oncogene 14: 1617-22; incorporate herein by reference in its entirety; herein incorporated by reference in its entirety.
Choong N W, Cohen E E (2006). Epidermal growth factor receptor directed therapy in head and neck cancer. Crit Rev Oncol Hematol 57: 25-43; incorporate herein by reference in its entirety; herein incorporated by reference in its entirety.
Chung C H, Ely K, McGavran L, Varella-Garcia M, Parker J, Parker N et al (2006). Increased epidermal growth factor receptor gene copy number is associated with poor prognosis in head and neck squamous cell carcinomas. J Clin Oncol 24: 4170-6; incorporate herein by reference in its entirety; herein incorporated by reference in its entirety.
Erjala K, Sundvall M, Junttila T T, Zhang N, Savisalo M, Mali P et al (2006). Signaling via ErbB2 and ErbB3 associates with resistance and epidermal growth factor receptor (EGFR) amplification with sensitivity to EGFR inhibitor gefitinib in head and neck squamous cell carcinoma cells. Clin Cancer Res 12: 4103-11; incorporate herein by reference in its entirety; herein incorporated by reference in its entirety.
Kamangar F, Dores G M, Anderson W F (2006). Patterns of cancer incidence, mortality, and prevalence across five continents: defining priorities to reduce cancer disparities in different geographic regions of the world. J Clin Oncol 24: 2137-50; incorporate herein by reference in its entirety; herein incorporated by reference in its entirety.
Lee L W, Taylor C E, Desaulniers J P, Zhang M, Hojfeldt J W, Pan Q et al (2009). Inhibition of ErbB2(Her2) expression with small molecule transcription factor mimics Bioorg Med Chem Lett 19: 6233-6; incorporate herein by reference in its entirety; herein incorporated by reference in its entirety.
Licitra L, Mesia R, Rivera F, Remenar E, Hitt R, Erfan J et al Evaluation of EGFR gene copy number as a predictive biomarker for the efficacy of cetuximab in combination with chemotherapy in the first-line treatment of recurrent and/or metastatic squamous cell carcinoma of the head and neck: EXTREME study. Ann Oncol; incorporate herein by reference in its entirety; herein incorporated by reference in its entirety.
Liu E, Thor A, He M, Barcos M, Ljung B M, Benz C (1992). The HER2 (c-erbB-2) oncogene is frequently amplified in in situ carcinomas of the breast. Oncogene 7: 1027-32; incorporate herein by reference in its entirety; herein incorporated by reference in its entirety.
Lo H W, Hung M C (2006). Nuclear EGFR signalling network in cancers: linking EGFR pathway to cell cycle progression, nitric oxide pathway and patient survival. Br J Cancer 94: 184-8; incorporate herein by reference in its entirety; herein incorporated by reference in its entirety.
Nicholson R I, Gee J M, Harper M E (2001). EGFR and cancer prognosis. Eur J Cancer 37 Suppl 4: S9-15; incorporate herein by reference in its entirety.
Oettgen P, Alani R M, Barcinski M A, Brown L, Akbarali Y, Boltax J et al (1997). Isolation and characterization of a novel epithelium-specific transcription factor, ESE-1, a member of the ets family. Mol Cell Biol 17: 4419-33; incorporate herein by reference in its entirety.
Oikawa T, Yamada T (2003). Molecular biology of the Ets family of transcription factors. Gene 303: 11-34; incorporate herein by reference in its entirety.
Rubin Grandis J, Methem M F, Gooding W E, Day R, Hoist V A, Wagener M M et al (1998). Levels of TGF-alpha and EGFR protein in head and neck squamous cell carcinoma and patient survival. J Natl Cancer Inst 90: 824-32; incorporate herein by reference in its entirety.
Santin A D, Bellone S, Roman J J, McKenney J K, Pecorelli S. (2008). “Trastuzumab treatment in patients with advanced or recurrent endometrial carcinoma overexpressing HER2/neu”. Int J Gynaecol Obstet 102 (2): 128-31; herein incorporated by reference in its entirety.
Schedin P J, Eckel-Mahan K L, McDaniel S M, Prescott J D, Brodsky K S, Tentler J J et al (2004). ESX induces transformation and functional epithelial to mesenchymal transition in MCF-12A mammary epithelial cells. Oncogene 23: 1766-79; incorporate herein by reference in its entirety.
Shimogawa H, Kwon Y, Mao Q, Kawazoe Y, Choi Y, Asada S et al (2004). A wrench-shaped synthetic molecule that modulates a transcription factor-coactivator interaction. J Am Chem Soc 126: 3461-71; incorporate herein by reference in its entirety.
Temam S, Kawaguchi H, El-Naggar A K, Jelinek J, Tang H, Liu D D et al (2007). Epidermal growth factor receptor copy number alterations correlate with poor clinical outcome in patients with head and neck squamous cancer. J Clin Oncol 25: 2164-70; incorporate herein by reference in its entirety.
Wang S C, Hung M C (2009). Nuclear translocation of the epidermal growth factor receptor family membrane tyrosine kinase receptors. Clin Cancer Res 15: 6484-9; incorporate herein by reference in its entirety.
Weihua Z, Tsan R, Huang W C, Wu Q, Chiu C H, Fidler I J et al (2008). Survival of cancer cells is maintained by EGFR independent of its kinase activity. Cancer Cell 13: 385-93; incorporate herein by reference in its entirety.
Semenza, G. L. Targeting HIF-1 for cancer therapy. Nat. Rev. Cancer 2003, 3, 721-732; herein incorporated by reference in its entirety.
Kersten, S.; Desvergne, B.; Wahli, W. Roles of PPARs in health and disease. Nature 2000, 405, 421-424; herein incorporated by reference in its entirety.
Rayet, B.; Gelinas, C. Aberrant rel/nflth genes and activity in human cancer. Oncogene 1999, 18, 6938-6947; herein incorporated by reference in its entirety.
Arndt, H.-D. Small Molecule Modulators of Transcription. Angew. Chem. 2006, 118, 4664-4673. Angew. Chem. Int. Ed. 2006, 45, 4552-4560; herein incorporated by reference in its entirety.
Mapp, A. K.; Ansari, A. Z. A TAD Further: Exogenous Control of Gene Activation. ACS Chem. Biol. 2007, 2, 62-75; herein incorporated by reference in its entirety.
Lee, L. W.; Mapp, A. K. Transcriptional Switches: Chemical Approaches to Gene Regulation. J. Biol. Chem. 2010, 285, 11033-11038; herein incorporated by reference in its entirety.
Fuxreiter, M.; Tompa, P.; Simon, I.; Uversky, V. N.; Hansen, J. C.; Asturias, F. J. Malleable machines take shape in eukaryotic transcriptional regulation. Nat. Chem. Biol. 2008, 4, 728-737; herein incorporated by reference in its entirety.
Sharma, D.; Fondell, J. D. Ordered recruitment of histone acetyltransferases and the TRAP/Mediator complex to thyroid hormoneresponsive promoters in vivo. Proc. Natl. Acad. Sci. U.S.A. 2002, 99, 7934-7939; herein incorporated by reference in its entirety.
Henchey, L. K.; Kushal, S.; Dubey, R.; Chapman, R. N.; Olenyuk, B. Z.; Arora, P. S. Inhibition of Hypoxia Inducible Factor 1—Transcription Coactivator Interaction by a Hydrogen Bond Surrogate α-Helix. J. Am. Chem. Soc. 2010, 132, 941-943; herein incorporated by reference in its entirety.
Moellering, R. E.; Cornejo, M.; Davis, T. N.; Bianco, C. D.; Aster, J. C.; Blacklow, S. C.; Kung, A. L.; Gilliland, D. G.; Verdine, G. L.; Bradner, J. E. Direct inhibition of the NOTCH transcription factor complex. Nature 2009, 462, 182-188; herein incorporated by reference in its entirety.
Best, J. L.; Amezcua, C. A.; Mayr, B.; Flechner, L.; Murawsky, C. M.; Emerson, B.; Zor, T.; Gardner, K. H.; Montminy, M. Identification of small-molecule antagonists that inhibit an activator: coactivator interaction. Proc. Natl. Acad. Sci. U.S.A. 2004, 101, 17622-17627.
Buhrlage, S. J.; Bates, C. A.; Rowe, S. P.; Minter, A. R.; Brennan, B. B.; Majmudar, C. Y.; Wemmer, D. E.; Al-Hashimi, H.; Mapp, A. K. Amphipathic small molecules mimic the binding mode and function of endogenous transcription factors. ACS Chem. Biol. 2009, 4, 335-344; herein incorporated by reference in its entirety.
Buhrlage, S. J.; Brennan, B. B.; Minter, A. R.; Anna, K. Stereochemical promiscuity in artificial transcriptional activators. J. Am. Chem. Soc. 2005, 127, 12456-12457; herein incorporated by reference in its entirety.
Lee, L. W.; Taylor, C. E. C.; Desaulniers, J. P.; Zhang, M.; Hojfeldt, J. W.; Pan, Q.; Mapp, A. K Inhibition of ErbB2 (Her2) expression with small molecule transcription factor mimics. Bioorg. Med. Chem. Lett. 2009, 19, 6233-6236; herein incorporated by reference in its entirety.
Rowe, S. P.; Casey, R. J.; Brennan, B. B.; Buhrlage, S. J.; Anna, K. Transcriptional up-regulation in cells mediated by a small molecule. J. Am. Chem. Soc. 2007, 129, 10654-10655; herein incorporated by reference in its entirety.
Slamon, D. J.; Godolphin, W.; Jones, L. A.; Holt, J. A.; Wong, S. G.; Keith, D. E.; Levin, W. J.; Stuart, S. G.; Udove, J.; Ullrich, A.; Press, M. F. Studies of the HER-2/neu proto-oncogene in human breast and ovarian cancer. Science 1989, 244, 707-712; herein incorporated by reference in its entirety.
Yarden, Y. Biology of HER2 and its importance in breast cancer. Oncology 2000, 61, 1-13; herein incorporated by reference in its entirety.
Slamon, D. J.; Clark, G. M.; Wong, S. G.; Levin, W. J.; Ullrich, A.; McGuire, W. L. Human breast cancer: correlation of relapse and survival with amplification of the HER-2/neu oncogene. Science 1987, 235, 177-182; herein incorporated by reference in its entirety.
Menendez, J. A.; Vellon, L.; Mehmi, I.; Oza, B. P.; Ropero, S.; Colomer, R.; Lupu, R. Inhibition of fatty acid synthase (FAS) suppresses HER2/neu (erbB-2) oncogene overexpression in cancer cells. Proc. Natl. Acad. Sci. U.S.A. 2004, 101, 10715-10720; herein incorporated by reference in its entirety.
Nahta, R.; Esteva, F. J. Trastuzumab: triumphs and tribulations. Oncogene 2007, 26, 3637-3643; herein incorporated by reference in its entirety.
Xia, W.; Gerard, C. M.; Liu, L.; Baudson, N. M.; Ory, T. L.; Spector, N. L. Combining lapatinib (GW572016), a small molecule inhibitor of ErbB1 and ErbB2 tyrosine kinases, with therapeutic anti-ErbB2 antibodies enhances apoptosis of ErbB2-overexpressing breast cancer cells. Oncogene 2005, 24, 6213-6221; herein incorporated by reference in its entirety.
Kong, A.; Calleja, V.; Leboucher, P.; Harris, A.; Parker, P. J.; Larijani, B. HER2 Oncogenic Function Escapes EGFR Tyrosine Kinase Inhibitors via Activation of Alternative HER Receptors in Breast Cancer Cells. PLoS One 2008, 3, e2881; herein incorporated by reference in its entirety.
Sergina, N. V.; Rausch, M.; Wang, D.; Blair, J.; Hann, B.; Shokat, K. M.; Moasser, M. M. Escape from HER-family tyrosine kinase inhibitor therapy by the kinase-inactive HERS. Nature 2007, 445, 437-441; herein incorporated by reference in its entirety.
Roe, S. M.; Prodromou, C.; O'Brien, R.; Ladbury, J. E.; Piper, P. W.; Pearl, L. H. Structural Basis for Inhibition of the Hsp90 Molecular Chaperone by the Antitumor Antibiotics Radicicol and Geldanamycin. J. Med. Chem. 1999, 42, 260-266; herein incorporated by reference in its entirety.
Isaacs, J. S.; Xu, W.; Neckers, L. Heat shock protein 90 as a molecular target for cancer therapeutics. Cancer Cell 2003, 3, 213-217; herein incorporated by reference in its entirety.
Berenbaum, M. C. What is Synergy? Pharmacological Reviews 1989, 41, 93-141; herein incorporated by reference in its entirety.
Borisy, A. A.; Elliott, P. J.; Hurst, N. W.; Lee, M. S.; Lehar, J.; Price, E. R.; Serbedzija, G.; Zimmermann, G. R.; Foley, M. A.; Stockwell, B. R.; Keith, C. T. Systematic discovery of multicomponent therapeutics. Proc. Natl. Acad. Sci. U.S.A. 2003, 100, 7977-7982; herein incorporated by reference in its entirety.
Petrov, K. G.; Zhang, Y. M.; Carter, M.; Cockerill, G. S.; Dickerson, S.; Gauthier, C. A.; Guo, Y.; Mook, R. A.; Rusnak, D. W.; Walker, A. L.; Wood, E. R.; Lackey, K. E. Optimization and SAR for dual ErbB-1/ErbB-2 tyrosine kinase inhibition in the 6-furanylquinazoline series. Bioorg. Med. Chem. Lett. 2006, 16, 4686-4691; herein incorporated by reference in its entirety.
Madhusudan, S.; Ganesan, T. S. Tyrosine kinase inhibitors in cancer therapy. Clinical Biochemistry 2004, 37, 618-635; herein incorporated by reference in its entirety.
M. C. Berenbaum, Pharmacological Reviews 1989, 41, 93-141; herein incorporated by reference in its entirety.
A. A. Borisy, P. J. Elliott, N. W. Hurst, M. S. Lee, J. Lehar, E. R. Price, G. Serbedzija,
G. R. Zimmermann, M. A. Foley, B. R. Stockwell, and C. T. Keith, Proceedings of the National Academy of Sciences of the United States of America 2003, 100, 7977-7982; herein incorporated by reference in its entirety.
H. Huezo, M. Vilenchik, N. Rosen, G. Chiosis, Chemistry & Biology, 2003, 10, 629-634; herein incorporated by reference in its entirety.
D. J. Slamon, W. Godolphin, L. A. Jones, J. A. Holt, S. G. Wong, D. E. Keith, W. J. Levin, S. G. Stuart, J. Udove, A. Ullrich, M. F. Press, Science 1989, 244, 707-712; herein incorporated by reference in its entirety.
A. A. Borisy, P. J. Elliott, N. W. Hurst, M. S. Lee, J. Lehar, E. R. Price, G. Serbedzija, G. R. Zimmermann, M. A. Foley, B. R. Stockwell, and C. T. Keith, Proc. Natl. Acad. Sci. U.S.A. 2003, 100, 7977-7982; herein incorporated by reference in its entirety.
K. G. Petrov, Y.-M. Zhang, M. Carter, G. S. Cockerill, S. Dickerson, C. A. Gauthier, Y. Guo, R. A. Mook, Jr., D. W. Rusnak, A. L. Walker, E. R. Wood, K. E. Lackey, Bioorganic & Medicinal Chemistry Letters 2006, 16, 4686-4691; herein incorporated by reference in its entirety.

### INCORPORATION B Y REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-39. (canceled)

40. A method for regulating ESX-mediated transcription of a gene of interest, comprising:
a) providing
i) host cells expressing: ESX, an ESX transcription coactivator protein required for said ESX-mediated transcription of a gene of interest, and a gene of interest, wherein ESX has a specific region where said ESX transcription coactivator protein binds; and
ii) small molecules capable of binding within said specific region;
b) delivering to said host cells an effective amount of said small molecules such that expression of said gene of interest is modified.

41. The method of claim 40,
wherein said ESX transcription coactivator protein required for said ESX-mediated transcription of a gene of interest is Med23,
wherein said gene of interest is selected from the group consisting of ErbB2(Her2) and EGFR, and
wherein said specific region is at least a portion of an eight amino acid (137-SWIIELLE-146) (SEQ ID NO:1) α-helical region in ESX reported to mediate the interaction between ESX and Med23.

42. The method of claim 40, wherein said host cells are cancer cells.

43. The method of claim 40, wherein said small molecules are isoxazolidine compounds.

44. The method of claim 43, wherein said isoxazolidine compounds are represented by the following formula: 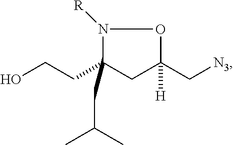
including salts, esters and prodrugs thereof, wherein R is a functional group that mimics at least a portion of an eight amino acid (137-SWIIELLE-146) (SEQ ID NO:1) α-helical region in ESX reported to mediate the interaction between ESX and Med23.

45. The method of claim 44,
wherein R is a functional group that mimics the effect of amino acid 138 within ESX, and/or
wherein R is a functional group that mimics the formation of a hydrophobic surface along an amphipathic helix within amino acids 137-146 of ESX.

46. The method of claim 44, wherein R is selected from the group consisting of:

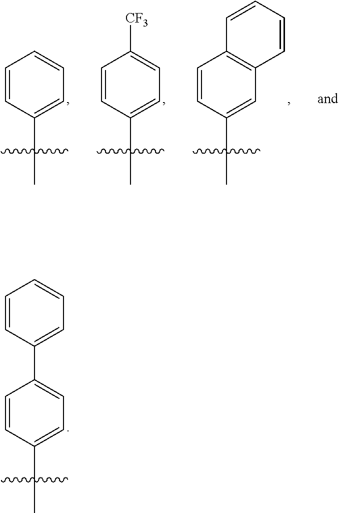

47. The method of claim 40, wherein said small molecules are selected from the group consisting of

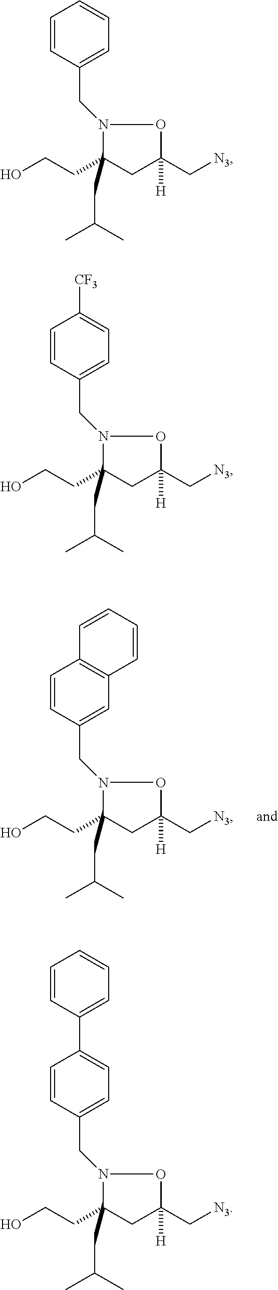

48. A method for treating a human subject having a disorder,
wherein said treating is selected from the group consisting of
administering to said human subject a pharmaceutical composition comprising an ESX-mediated transcription inhibitor, wherein said disorder is a disorder having elevated EGFR expression, and
co-administering to said subject an ESX-mediated transcription inhibitor and one or more agents known to target the activity and lifetime of an erbB2 oncoprotein, wherein said disorder is a disorder having elevated erbB2 expression.

49. The method of claim 48, wherein said disorder is cancer.

50. The method of claim 48,
wherein said disorder is a disorder having elevated EGFR expression, wherein said disorder is HNSCC, or
wherein said disorder is a disorder having elevated erbB2 expression, wherein said disorder is breast cancer, stomach cancer, ovarian cancer, or endometrial cancer.

51. The method of claim 48, wherein said ESX-mediated transcription inhibitor is an isoxazolidine compound.

52. The method of claim 51, wherein said isoxazolidine compounds is represented by the following formula: 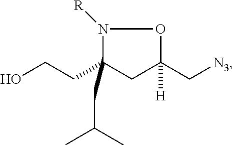
including salts, esters and prodrugs thereof, wherein R is a functional group configured to mimic at least a portion of an eight amino acid (137-SWIIELLE-146) (SEQ ID NO:1) α-helical region in ESX.

53. The method of claim 52,
wherein R is a functional group that mimics the effect of amino acid 138 within ESX, and/or
wherein R is a functional group that mimics the formation of a hydrophobic surface along an amphipathic helix within amino acids 137-146 of ESX.

54. The method of claim 52, wherein R is selected from the group consisting of:

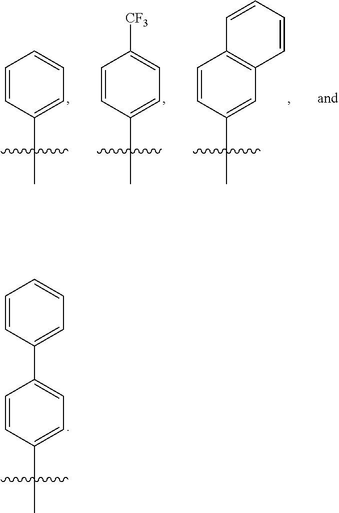

55. The method of claim 48, wherein said small ESX-mediated transcription inhibitor is selected from the group consisting of

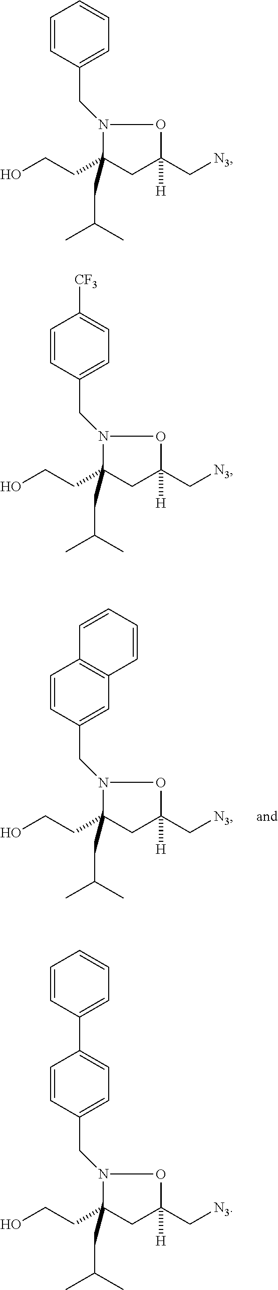

56. The method of claim 48, further comprising co-administering to the subject effective amounts of one or more therapeutic agents selected from the group consisting of cetuximab, panitumumab, zalutumubab, nimotuzumab, matuzumab gefitinib, afatinib, erlotinib, and lapatinib.

57. The method of claim 48, wherein said one or more agents known to target the activity and lifetime of an erbB2 oncoprotein is selected from the group consisting of a tyrosine kinase inhibitor and an anti-tumor antibiotic.

58. The method of claim 57,
wherein said tyrosine kinase inhibitor is selected from the group consisting of afatinib, gefitinib, erlotinib, and lapatinib, and
wherein said anti-tumor antibiotic is selected from the group consisting of geldanamycin, 17-N-Allylamino-17-demethoxygeldanamycin (17-AAG), and 17-Dimethylamino ethylamino-17-demethoxygeldanamycin (17-DMAG).

59. A method for identifying ESX-mediated transcription modulators, comprising:
a) providing i) host cells expressing ESX, a gene whose transcription is regulated by ESX, and ESX-mediated transcription coactivating compounds required for said ESX-mediated transcription of said gene of interest, and ii) a potential ESX-mediated transcription modulator,
b) delivering to the host cells an effective amount of the potential ESX-mediated transcription modulator, and
c) detecting changes in ESX-mediated transcription, wherein inhibition in ESX-mediated transcription indicates said potential ESX-mediated transcription modulator is an ESX-mediated transcription inhibitor, wherein enhancement in ESX-mediated transcription indicates said potential ESX-mediated transcription modulator is an ESX-mediated transcription enhancer.


SEQUENCE LISTING (1 sequence)

SEQ ID NO: 1 - protein, 8 aa, Homo sapiens
SWIIELLE